SEQUENCE LISTING (14 sequences)

SEQ ID NO: 1 - DNA, 666 bp, Paenibacillus polymyxa
atgacttcgattaaacagcttacattgtatacggccgagcttgaccggatgctagcattttatacgaatatgcttggtgcgcagcatgtgcatgagcaagcagatgcgtttacgatccagctaggagtatcacagattcaatttcgtgcagctgctgatggaacaaagcccttttaccatattgctatcaatatcgcggcaaaccattttcaagagggaaaagcctggctcagcggctttggtgaattgctaacggaaaatgatgaagatcaggcatactttcccttctttaacgcgtactcctgttatgtagaagacccgtctggtaatattattgaactcatctcgcgtcagcaagctgcacctgtactggataagcccttctcagcggatcagctactaagcatcggtgagattaatataacaaccagcgatgtagagcaagctgcaacacgattaaagcaagcagaactgcctgtaaagctagaccagattgagccagcaggcttaaattttatcggtgatcaggatttgttcctgctgctgggtcctccaggacgacgctggttattttcagaacgcgtagccgtgatctatccgttacagatggagctggataacggcgtcagtctggcgattacagagacaggtgaactggtgatctaa

SEQ ID NO: 2 - protein, 221 aa, Paenibacillus polymyxa
MTSIKQLTLYTAELDRMLAFYTNMLGAQHVHEQADAFTIQLGVSQIQFRAAADGTKPFYHIAINIAANHFQEGKAWLSGFGELLTENDEDQAYFPFFNAYSCYVEDPSGNIIELISRQQAAPVLDKPFSADQLLSIGEINITTSDVEQAATRLKQAELPVKLDQIEPAGLNFIGDQDLFLLLGPPGRRWLFSERVAVIYPLQMELDNGVSLAITETGELVI

SEQ ID NO: 3 - DNA, 20 bp, Artificial Sequence
  Primer
agagtttgatcmtggctcag

SEQ ID NO: 4 - DNA, 19 bp, Artificial Sequence
  Primer
ggytaccttgttacgactt

SEQ ID NO: 5 - DNA, 20 bp, Artificial Sequence
  Primer
aattaaccctcactaaaggg

SEQ ID NO: 6 - DNA, 22 bp, Artificial Sequence
  Primer
gtaatacgactcactatagggc

SEQ ID NO: 7 - DNA, 19 bp, Paenibacillus polymyxa
gaactcatctcgcgtcagc

SEQ ID NO: 8 - DNA, 19 bp, Paenibacillus polymyxa
gcagcttgctctacatcgc

SEQ ID NO: 9 - DNA, 26 bp, Paenibacillus polymyxa
catatgacttcgattaaacagcttac

SEQ ID NO: 10 - DNA, 24 bp, Paenibacillus polymyxa
ggatccttagatcaccagttcacc

SEQ ID NO: 11 - DNA, 29 bp, Artificial Sequence
  primer
ctcgagatgacttcgattaaacagcttac

SEQ ID NO: 12 - DNA, 24 bp, Artificial Sequence
  primer
ctcgagttagatcaccagttcacc

SEQ ID NO: 13 - DNA, 21 bp, Artificial Sequence
  primer
tgcagctgctgatggaacaaa

SEQ ID NO: 14 - DNA, 21 bp, Artificial Sequence
  primer
ttatccagtacaggtgcagct

(19) United States
(12) Patent Application Publication
(10) Pub. No.: US 2010/0269215 A1
(43) Pub. Date: Oct. 21, 2010

(54) Tfla gene which can degrade toxoflavin and its chemical derivatives and transgenic organisms expressing tfla gene

(75) Inventors: In Gyu Hwang, Seoul (KR); Jae Sun Moon, Daejeon (KR); Nam Soo Jwa, Seoul (KR)

Correspondence Address: OSHA LIANG L.L.P., TWO HOUSTON CENTER, 909 FANNIN, SUITE 3500, HOUSTON, TX, 77010, US

(73) Assignee: SNU R&DB Foundation, Seoul (KR)

(21) Appl. No.: 12/308,524

(22) PCT Filed: Jun. 21, 2007
(86) PCT No.: PCT/KR2007/003010

§ 371 Date: Dec. 17, 2008

(30) Foreign Application Priority Data
Jun. 21, 2006 (KR) 10-2006-0055863
Dec. 5, 2006 (KR) 10-2006-0121972

(51) Int. Cl. C12N 15/82; C12N 5/10; A01H 5/00; A01H 5/10

(52) U.S. Cl. 800/278; 435/320.1; 435/419; 800/298; 800/320.2

(57) ABSTRACT

An expression cassette of selection marker for plant transformation includes the following sequences that are operably linked in a 5′ to 3′ direction: (i) a promoter sequence; (ii) a coding sequence for an enzyme which degrades toxoflavin; and (iii) a 3′-untranslated terminator sequence. A method of producing a transgenic plant using the above-describe expression cassette includes (i) transforming plant cells with a recombinant vector that includes the above-described expression cassette to produce transgenic plant cells; (ii) proliferating said transgenic plant cells in a media comprising toxoflavin to produce selected transgenic plant cells; and (iii) growing a transgenic plant from said selected transgenic plant cells.

### BRIEF DESCRIPTION OF DRAWINGS

FIG. 1 represents a schematic diagram for isolation and characterization of Paenibacillus polymyxa JH2 from mountain soil, rice paddy or field soil, or rice seeds, etc.

FIG. 2 represents degradation of toxoflavin by E. coli HB101 which carries cosmid clone of Paenibacillus polymyxa JH2 (all the clones showed 1.5 kb EcoRI fragment).

FIG. 3 shows the result of similarity analysis for the proteins of ring-cleavage extradiol dioxygenase of Exiguobacterium sp. 255-15, with ((2) a unknown protein of Bacillus halodurans C-125, (3) a unknown protein of Bacillus halodurans C-125, (4) NahC of Bacillus sp. JF8, (5) NahH of Bacillus sp. JF8, (6) ThnC of Sphingopyxis macrogoltabida TFA, (7) BphC of Pseudomonas sp. LB400, (8) conserved hypothetical protein of X. axonopodis pv. citri str. 306 (9) conserved protein between the two peptides is marked with *).

FIG. 4 shows the purified TflA protein, in which (A) is a Coomassie staining of TflA protein, (B) is a result of TLC plate analysis for determining the effect by Mn⁺⁺ and DTT on degradation of toxoflavin by TflA (100 μM of toxoflavin was comprised in every lane: lane 1, 1 mM MnCl₂; lane 2, purified His-TflA and 1 mM MnCl₂; lane 3, 5 mM DTT; lane 4, purified His-TflA and 5 mM DTT; lane 5, 5 mM DTT/1 mM MnCl₂; lane 6, purified His-TflA plus and DTT/1 mM MnCl₂).

FIG. 5(A) shows an optimum temperature for purified His-TflA to degrade toxoflavin and FIG. 5(B) shows the level of toxoflavin degradation by His-TflA with the lapse of time.

FIG. 6 shows an optimum pH for purified His-TflA to degrade toxoflavin.

FIG. 7 shows a chemical structure of toxoflavin and its derivatives (circle represents a different kind of functional groups).

FIG. 8 shows a degradation of toxoflavin and its derivatives by His-TflA.

FIG. 9 is a Lineweaver-Burk plot which indicates the degradation of toxoflavin and its derivatives by His-TflA.

FIG. 10(A) is a diagram showing the genetic structure of pCamLA gene and FIG. 10(B) is a diagram showing the genetic structure of pJ904(pCamLA::tflA) (MCS, multiple cloning site; LB, left border; RB, right border; 35S, 35S promoter; P. PstI; Sm, SmaI; S, SacI),

FIG. 11 includes images for preparing transgenic rice plant.

FIG. 12(A) is a diagram showing the genetic structure of pJ904 gene (pCamLA::tflA) while FIG. 12(B) shows a result of Southern blot analysis for the transgenic rice ((A) T-DNA region of pJ904. LB, left border; RB, right border; Hyg^{R}, hygromycin phosphotransferase; 35S, CaMV 35S promoter. (B) Southern blot analysis of transgenic rice T2 plants. M, Molecular size marker; 1, Cv6-2; 2, Dt1-1; 3, Dt1-2; 4, Dt3-6; 5, Dt27-1; 6, Dt27-2; 7, Dt27-3; 8, Dt27-4; 9, Dt34-1; 10, Dt34-3; 11, Ct36-3; 12, pJ904; 13, Cv10-1; 14, Dt4-1; 15, Dt4-3; 16, Dt7-7; 17, Dt19-5; 18, Dt40-5; 19, Ct18-2; 20, Ct18-4; 21, Ct18-5; 22, Ct18-6; 23, Ct9-1; 24, pJ904; 25, Cv6-4; 26, Dt2-1; 27, Dt2-5; 28, Dt7-3; 29, Dt7-5; 30, Dt7-9; 31, Dt7-10; 32, Dt9-6; 33, Dt16-1; 34, Dt19-3; 35, Dt38-3; 36, pJ904),

FIG. 13 shows a result of Southern blot analysis for wild type rice and the transgenic T2 plant (M, Marker; WT, Dongjinbyeo wild-type plant; Dt, Dongjinbyeo transgenic T2 plants expressing tflA; TflA, purified His6-tagged TflA).

FIG. 14 includes the photo images taken for pieces of rice leaf which were treated with toxoflavin (A, Dongjinbyeo wild-type plant; B and C, Individual transgenic lines (B, Dt40-6; C, Dt19-5); D, Dongjinbyeo wild-type plant in the dark),

FIG. 15 is a schematic diagram of T-DNA comprised in pCamLA.

FIG. 16 is a schematic diagram of T-DNA comprised in pTflA.

FIG. 17 includes the photo images taken for rice callus that was transformed with either pCamLA (left) or pTflA (right) vector followed by the second selection with hygromycin (3 ug/ml).

FIG. 18 includes the photo images taken for rice callus that was transformed with either pCamLA (left) or pTflA (right) vector followed by the selection with toxoflavin (5 ug/ml), in which said selection is carried out before redifferentiation of the rice callus.

FIG. 19 includes the photo images taken for rice callus that was transformed with pCamLA followed by the selection with toxoflavin (7.5 ug/ml), in which said selection is carried out four weeks after placing the rice callus to medium for redifferentiation.

FIG. 20 includes the photo images taken for rice callus that was transformed with pTflA followed by the selection with toxoflavin (7.5 ug/ml), in which said selection is carried out four weeks after placing the rice callus to medium for redifferentiation.

FIG. 21 shows the result of agarose electrophoresis of PCR product which is obtained from PCR amplification of genomic DNA isolated from selected transgenic organisms.

FIG. 22 shows transgenic plants in which vectors of pCamLA:tflA, pCamLA(□hpt):tflA, pMBP1:tflA and pMBP1 are inserted, respectively.

FIG. 23 shows the result of agarose electrophoresis of PCR product which is obtained from PCR amplification of genomic DNA isolated from selected transgenic organisms.

FIG. 24 shows a bleaching effect tested for transgenic plants.

### DETAILED DESCRIPTION OF THE INVENTION

### Purpose of the Invention

### Technical Field of the Invention and Background Art

The present invention relates to a microorganism which can degrade toxoflavin and its derivatives, a protein which can degrade toxoflavin and its derivatives, a use of said protein as a selection marker for transformation of plants, a gene which encodes said protein, a recombinant expression vector comprising said gene, a transgenic organism which is transformed with said vector, an expression cassette of a selection marker comprising tflA gene for plant transformation, a recombinant vector comprising said expression cassette, a plant which is transformed with said vector, a method of selecting transgenic plants using tflA gene, and a method of preparing transgenic plants using tflA gene.

Rice grain rot is caused by gram negative bacteria called Burkholderia glumae and is very sensitive to change in weather. Recently it draws an attention in rice cultivating countries, including Korea, Japan, countries of South East Asia and America. It has been reported that rice grain rot spreads during flowering season of rice plants, which is high in both of temperature and humidity, and can cause about 34% drop in crop harvest. It has been also reported that Burkholderia glumae produce toxoflavin, reumycin and fervenulin, which are essential pathogenic elements for outbreak of rice grain rot and bacterial blight. Toxoflavin is known as the most critical pathogenic element among them.

‘Paenibacillus polymyxa’, which usually thrives near roots of a plant, can promote growth of the plant and prevent an occurrence of plant diseases while interacting with other microorganisms in soil. Further, producing various kinds of antibiotic substance and hydrolyzing enzyme, it is regarded as a beneficial microorganism. Still further, found to be a gram positive bacteria which can fix nitrogen, its importance is being noticed more and more recently.

An expression vector comprises at least one genetic marker which can be used for selection of transformed cells by inhibiting growth of the cells which do not comprise a selection marker gene. Most of selective marker genes that are used for transformation of plant have been isolated from bacteria, and they encode an enzyme which can metabolically degrade selective chemicals, that can be either antibiotics or herbicides.

The most widely used selection marker genes for the transformation of plant is neomycin phosphotransferase II (nptII) that is isolated from Tn5. Others include hygromycin phosphotransferase gene which confers resistance to one antibiotic, hygromycin.

Many of the selection markers have been used for selection of transgenic plant tissues. However, such selection system based on the use of toxic chemicals carries a shortcoming or a limit. First, direct recovery of normal, viable transgenic plants using a chemical selection method can be difficult. Second, not all of the selection marker systems can be applied to every tissue and every kind of plants. Third, some of chemicals which need to be added for successful selection are antibiotics. Propagation of genes that are resistant to antibiotics and herbicides should be prevented as much as possible in order to avoid a risk of conferring resistance to any pathogens. Fourth, because some of the chemicals which need to be added for successful selection are quite expensive, there is a need for development of cheaper selection markers.

The present invention is to provide tflA protein of Paenibacillus polymyxa JH2 and genes encoding said protein, which is involved with resistant reaction to rice grain rot, to identify the characteristics of said tflA protein, to produce transgenic rice plant through recombination of said gene, and to provide high quality and non-toxic rice plant that has improved resistance to blight and harmful insects which cause rice grain rot. Furthermore, the present invention is to provide a selection marker for easy and convenient selection of transgenic plants, using tflA enzyme which can degrade toxoflavin.

### Technical Subject to be Achieved by the Invention

Inventors of the present invention prepared a transgenic organism which shows resistance to rice grain rot by expressing tflA protein of Paenibacillus polymyxa JH2 that is related to resistance to rice grain rot. Understanding the interaction between the organism and Paenibacillus polymyxa JH2, the inventors were able to provide a new system for controlling the plant disease. As a result, the present invention was completed.

Thus, one object of the present invention is to provide a microorganism which can degrade toxoflavin and its derivatives.

Another object of the present invention is to provide tflA protein which can degrade toxoflavin and its derivative.

Another object of the present invention is to provide a use of tflA protein as a selection marker for transformation of plants.

Another object of the present invention is to provide a gene encoding tflA protein, that can degrade toxoflavin and its derivatives.

Another object of the present invention is to provide a recombinant expression vector comprising tflA gene.

Another object of the present invention is to provide recombinant tflA protein which is expressed by the recombinant expression vector comprising tflA gene.

Another object of the present invention is to provide a transgenic organism which is transformed with said recombinant expression vector comprising tflA gene.

Another object of the present invention is to provide an expression cassette of a selection marker comprising tflA gene for plant transformation.

Another object of the present invention is to provide a recombinant vector comprising said expression cassette.

Another object of the present invention is to provide a plant which is transformed with said vector.

Another object of the present invention is to provide a method of selecting transgenic plants using tflA gene.

Another object of the present invention is to provide a method of preparing transgenic plants using tflA gene.

### Constitution of Invention

To achieve the object of the invention, the present invention provides a microorganism which can degrade toxoflavin and its derivatives. Preferably, the microorganism has bacterial origin. More preferably, it is from genus Paenibacillus, still more preferably it is Paenibacillus polymyxa and still further more preferably it is Paenibacillus polymyxa JH2, which has been deposited with Korean Bioengineering Institute on Jun. 13, 2006 (Deposit No. KCTC 10959BP). Toxoflavin is an essential pathogenic element causing rice grain rot and bacterial blight in field crops. According to the present invention, derivatives of toxoflavin include any derivatives which have the same activity as toxoflavin. Said derivatives include 3-methyltoxoflavin, 4,8-dihydrotoxoflavin and 3-methylreumycin, etc., but are not limited thereto.

Paenibacillus polymyxa, which usually thrives near roots of plant, promotes growth of the plant and prevents plant diseases by interacting with other microorganisms in soil. Further, producing various kinds of antibiotic substance and hydrolyzing enzyme, it is categorized as a beneficial microorganism. The inventors of the present invention found that tflA protein from Paenibacillus polymyxa JH2 degrades toxoflavin, which is a substance causing rice grain rot.

Further, the present invention is to provide tflA protein which can degrade toxoflavin and derivatives thereof. Variants of the gene which encode said protein are also within the scope of the present invention. The variants may have different amino acid sequence but have a similar functional and immunological characteristic compared to the amino acid sequence of SEQ ID NO: 2. Variant proteins may comprise a sequence which has sequence homology of at least 50%, preferably at least 70%, more preferably at least 80%, still more preferably at least 90%, and still further more preferably at least 95%, compared to the amino acid sequence of SEQ ID NO:2. Most preferably, said variant protein may comprise the amino acid sequence of SEQ ID NO: 2. It may also comprise a sequence in which one or more amino acid residues of the sequence of said protein are substituted, inserted, or deleted to maintain the ability of degrading toxoflavin. Method of substituting, inserting or deleting amino acid residues can be any method that is known to a skilled person in the pertinent art.

According to one embodiment of the present invention, the above-described tflA protein can be used as a selection marker for plant transformation. tflA enzyme of the present invention which can degrade toxoflavin confers resistance to the chemical compound of toxoflavin. Toxoflavin is the most important pathogenic element which causes rice grain rot. Transgenic organism of the present invention can be a plant. Preferably, it can be either rice plant or Arabidopsis thaliana.

The present invention also provides a gene which encodes tflA protein. Preferably, such gene is a gene comprising the nucleotide sequence of SEQ ID NO: 1. Such gene may comprise a nucleotide sequence which has sequence homology of at least 50%, preferably at least 70%, more preferably at least 80%, still more preferably at least 90%, and still further more preferably at least 95%, compared to the nucleotide sequence of SEQ ID NO: 1. Genes having such sequence homology can be prepared by substituting, inserting or deleting the nucleotide sequence of SEQ ID NO: 1. Method of substituting, inserting or deleting nucleotides can be any method that is known to a skilled person in the pertinent art. Meanwhile, the protein coded by said gene variants with substitution, insertion or deletion of nucleotides should maintain the ability of degrading toxoflavin.

“Percentage (%) of sequence homology” can be determined by comparing the two sequences of interest that are aligned optimally to each other with a comparative region. A part of polynucleotide and polypeptide sequences of the comparative region may comprise an addition or a deletion (i.e., a gap), compared to a reference sequence relating to the optimally aligned two sequences (without addition or deletion). Said percentage is based on the calculation which comprises determining the number of location in which the same nucleotides or amino acid residues are present for both of the sequences, obtaining the number of matching location therefrom, and dividing the number of matching location by the total number of location present in the comparative region and then multiplying thus obtained value with 100 to have the percentage (%) of sequence homology. The best-optimized alignment of sequences for comparison can be carried out by an implementation by computer using a known operating method (GAP, BESTFIT, FASTA and TFAST in the Wisconsin Genetics Software Package, Genetics Computer Group (GCG), 575 Science Dr., Madison, Wis., or BlastN and BlastX available from the National Center for Biotechnology Information) or by a determination.

Terms of “substantially identical” or “substantially similar” mean that the polypeptide with such characteristic comprises a sequence which can be hybridized with a target polypeptide under a stringent condition. Here, stringent condition indicates a condition with 2× SSC solution and temperature of 65□.

“Substantially similar” polypeptides share the above-described sequence except that a location of residues that is not the same for two sequences can be different due to a conservative change in amino acids. The conservative change in amino acids indicates mutual exchange among amino acid residues having a similar side chain. For example, a group of amino acid having an alkyl side chain includes glycine, alanine, valine, leucine and isoleucine, a group of amino acid having a hydroxyl side chain includes serine and threonine, a group of amino acid having an amide side chain includes asparagines and glutamine, a group of amino acid having an aryl side chain includes phenylalanine, tyrosine, and tryptophan, a group of amino acid having a basic side chain includes lysine, arginine, and histidine, and a group of amino acid having a sulfur-comprising side chain includes cysteine and methionine.

Substantially identical polynucleotide sequence indicates that the polynucleotide of interest comprises a nucleotide sequence that is at least 70% identical, preferably at least 80%, more preferably at least 90%, and the most preferably at least 95% identical. Another meaning of being substantially identical is that, when two nucleotide molecules are hybridized specifically to each other under stringent condition, their sequences are substantially identical to each other. The stringent condition varies depending on nucleotide sequence. Thus, it can be different at different condition. Generally, at certain ionic strength and pH, the stringent condition is selected to have a temperature that is about 10□ lower than heat-melting point (Tm) of a specific sequence. Tm is defined as a temperature at which 50% of a target sequence is hybridized to a fully complementary probe (under the condition of certain ionic strength and pH). Tm, which is determined by length and composition of nucleotide bases of a probe, can be calculated using teachings described in the literature (see, Sambrook, T. et al., (1989) Molecular Cloning—A Laboratory Manual (second edition), Volume 1-3, Cold Spring Harbor Laboratory, Cold Spring). Typically the stringent condition for carrying out Southern blot analysis includes washing with 0.2×SSC at 65□. For an appropriate oligonucleotide probe, washing is typically carried out with 6×SSC at 42□.

The present invention further provides a recombinant expression vector comprising the above-described tflA gene. Preferably, such vector corresponds to a vector that can be expressed in E. coli, virus, plant or animal. In one embodiment, the present invention provides tflA expression vector which is prepared by incorporating tflA gene from Paenibacillus polymyxa JH2 to pCamLA vector, in which hygromycin phosphotransferase Hyg^{R} is comprised inside T-DNA while a kanamycin resistant gene is comprised outside T-DNA.

“Vector” is a vehicle for transferring nucleic acids into a host cell. Vector can be a replicon to which other DNA fragment is attached so that the attached fragment can be replicated. “Replicon” functions as an individual unit of DNA replicon in living organism and corresponds to a genetic element which can replicate with self-control (e.g., plasmid, phage, cosmid, chromosome, and virus). “Vector” is defined as a means for introducing nucleic acids into a cell, either in vivo or in vitro. It includes viral and non-viral ones. Viral vector includes, retrovirus, adeno-realted virus, baculovirus, herpes simplex, vaccinia, Epstein-Barr and adenovirus vector, etc. Non-viral vector includes, plasmid, liposome, electrically charged lipids (cytofectin), DNA-protein complex and biopolymers, etc. In addition to nucleic acids, vector comprises at least one regulatory region and/or selection marker, which is useful for screening, detecting and monitoring the result of the nucleic acid transfer (e.g., transfer to a certain tissue and continued expression, etc.).

The present invention further provides the recombinant tflA protein which is expressed by the recombinant expression vector of the present invention. While the recombinant tflA protein expressed in E. coli is not glycosylated, the recombinant tflA protein expressed in plant or animal cell is glycosylated. Thus, depending on the intended use of the protein, a suitable recombinant tflA protein can be chosen and be used.

The present invention further provides the transgenic organism that is transformed with the recombinant expression vector of the present invention. Said transgenic organism can be a microorganism, virus, a plant or an animal, etc. Preferably, it is a plant, and more preferably it is rice plant.

The present invention further provides a method of preparing transgenic rice plant which can be asexually reproduced by tissue culture, characterized in that tflA gene is expressed from tflA expression vector, which is prepared by incorporating tflA gene from Paenibacillus polymyxa JH2, that can degrade toxoflavin causing rice grain rot, to pCamLA vector wherein hygromycin phosphotransferase Hyg^{R} is comprised inside T-DNA while a kanamycin resistant gene is comprised outside T-DNA.

The present invention further provides the transgenic rice plant which can be asexually reproduced by tissue culture, characterized in that tflA gene from Paenibacillus polymyxa JH2, which can degrade toxoflavin causing rice grain rot, is expressed so that the transgenic plant can have resistance to rice grain rot.

The present invention further provides the expression cassette of selection marker for plant transformation, comprising the following sequences that are operably linked in 5′ to 3′ direction:
(i) a promoter sequence;
(ii) a coding sequence for the enzyme which can degrade toxoflavin; and
(iii) a 3′-untranslated terminator sequence.

To make it possible that a protein is expressed in a host cell in a way that it can confer resistance to a formulation with selective toxicity, the coding sequence for the enzyme which can degrade toxoflavin is generally provided as an expression cassette having a regulatory element which enables the recognition of the coding sequence by biochemical machinery of the host cell and the transcription and the translation of its open reading frame in the host cells. The expression cassette generally includes not only an initiation region for transcription which can be appropriately derived from any gene that can be expressed in the host cell, but also other initiation region for transcription that is intended for recognition and attachment by ribosomes. In eukaryotic plant cells, the expression cassette usually further comprises a termination region for transcription located downstream of said open reading frame, in order to achieve the termination of the transcription and the polyadenylation of primary transcript. Moreover, an amount of codon usage can be suitable for the amount of codon usage allowed in the host cell. The basic principle which determines the expression of hybrid DNA construct in selected host cell is generally understood by a skilled person in the art, and the preparational method of hybrid DNA construct that is to be expressed is common for any kind of host cells including prokaryotes and eukaryotes.

For the expression cassette according to one embodiment of the present invention, the above-described promoter can be CaMV 35S, actin, ubiquitin, pEMU, MAS or histone promoter, but is not limited thereto. The term “promoter” indicates a DNA region located upstream of the structural sequence and it refers to DNA molecule at which RNA polymerase binds to initiate transcription. “Plant promoter” indicates a promoter which can initiate transcription in plant cells. “Constructive promoter” indicates a promoter which is active in most of environmental and developmental conditions and also under division of the cells. Because the selection of transgenic organism can be carried out by different tissues at different stage, a constructive promoter can be preferable in the present invention. Therefore, the constructive promoter does not limit the possibility of selection.

For the expression cassette according to one embodiment of the present invention, the above-described terminator can be nopaline synthase (NOS) or rice α-amylase RAmyl A terminator, but is not limited thereto. Regarding the necessity of terminator, it is generally known that reliability and efficiency of transcription in plants are increased by the presence of terminator. Thus, in view of the context of the present invention, it is highly preferable to use such terminator.

For the expression cassette according to one embodiment of the present invention, the above-described coding sequence for the enzyme which can degrade toxoflavin may include the nucleotide sequence of SEQ ID NO: 1. In addition, it may comprise a nucleotide sequence which has sequence homology of at least 70%, more preferably at least 80%, still more preferably at least 90%, and most preferably at least 95%, compared to the sequence of SEQ ID NO: 1.

In the present invention the term “operably linked” indicates the element of the expression cassette which functions as a unit to express a heterogeneous protein. For instance, a promoter operably linked to a heterogeneous DNA which encodes a protein promotes the production of functional mRNA corresponding to the heterogeneous DNA.

For the expression cassette according to one embodiment of the present invention, an expression cassette for target protein comprising (i) a promoter sequence, (ii) a coding sequence for the target protein, and (iii) a 3′-untranslated terminator sequence can be further included. The target protein includes commercially available therapeutic proteins and polypeptides such as erythropoietin (EPO), tissue plasminogen activator (t-PA), urokinase and prourokinase, growth hormone, cytokine, Factor VIII, epoetin-α, granulocyte colony stimulating factor and vaccine, etc., but is not limited thereto.

For the expression cassette according to one embodiment of the present invention, the expression cassette for target protein can be constructed as a single expression cassette wherein it is in tandem array with the above-described expression cassette for selection marker. In other words, the expression cassette for target protein and the expression cassette for selection marker can be lying one after the other in a sequence. In addition, it is also possible to have the expression cassette for target protein and the above-described expression cassette for selection marker in separate expression cassettes.

The present invention further provides the recombinant vector which comprises the expression cassette of the present invention. In order to have an open reading frame maintained in host cells, the recombinant vector will be provided in a form of replicon which comprises the open reading frame of the present invention that is linked to DNA to be recognized and replicated by the host cells that are selected. Therefore, choice of replicon greatly depends on the selected host cells. Making a choice for replicon that is suitable for the selected host cells is well within the skill of a person in the pertinent art.

A specific type of replicon can transfer the whole or a part of itself to other cells such as plant cells. As a result, the open reading frame of the present invention can be simultaneously transferred to the plant cells. Replicon having such activity is referred to as a “vector” in the present invention. Examples of such vector include Ti-plasmid vector, which can transfer a part of itself (so called T-region) to plant cells when it is present in an appropriate host cells such as Agrobacterium tumefaciens. Another type of Ti-plasmid vector is used for transferring DNA of existing plant cells or its hybrid DNA to protoplast in which said DNA sequences are appropriately introduced to the genome of the plant to produce a new kind of plant (see, EP 0 116 718 B1). An especially preferred type of Ti-plasmid vector is so-called binary vector as described in EP 0 120 516 B1 and U.S. Pat. No. 4,940,838. Another type of vector that can be used for introducing the DNA of the present invention to plant host cells may be chosen from viral vectors originating from double stranded plant virus (e.g. CaMV), single stranded virus or Gemini virus, etc., for example an incomplete plant viral vector. Use of such vectors can be especially advantageous when an appropriate transformation of plant host cells is not easy. Examples of such plant may include lignum sp., particularly trees and vine plants.

In order to achieve another purpose of the present invention, host cells that are transformed with the recombinant vector of the present invention are provided. Preferably, said host cells may belong to Agrobacterium sp. More preferably, it may be Agrobacterium tumefaciens.

In order to achieve another purpose of the present invention, plants that are transformed with the recombinant vector of the present invention are provided. The plants can be either rice plant or Arabidopsis thaliana.

Transformation of plants includes any method of transferring DNA to plants. Such method for transformation does not necessarily require a period for tissue culture and/or reproduction. Transformation of plant species is now common not only for plants of dicotyledonea but also for plants of monocotyledonea. In principle, any method for plant transformation can be used for introducing hybrid DNA of the present invention to appropriate progenitor cells. Any method chosen from the following can be appropriately used; calcium/polyethylene glycol method (Krens, F. A. et al., 1982, Nature 296, 72-74; Negrutiu I. et al., June 1987, Plant Mol. Biol. 8, 363-373), electroporation of protoplast (Shillito R. D. et al., 1985 Bio/Technol. 3, 1099-1102), microinjection into plant elements (Crossway A. et al., 1986, Mol. Gen. Genet. 202, 179-185), particle bombardment of various plant elements (DNA or RNA-coated particles) (Klein T. M. et al., 1987, Nature 327, 70), infection with (incomplete) virus for gene transfer mediated by Agrobacterium tumefaciens using infiltration of plants or transformation of ripe pollen or microspore (EP 0 301 316), etc. In the present invention, preferred method includes DNA transfer mediated by Agrobacterium. Particularly preferred method is the one using so-called binary vector as described in EP A 120 516 and U.S. Pat. No. 4,940,838.

For achieving another object of the invention, transgenic seeds that are transformed with the recombinant vector of the present invention are provided.

For achieving still another object of the invention, a method of selecting transgenic plants comprising the following steps is provided:

carrying out the transformation of a plant, a part of plant, or the plant cells with the recombinant vector of the present invention; and

amplifying the resulting transgenic organism in the media containing toxoflavin. The method of the present invention comprises a step of carrying out the transformation of a plant, a part of plant, or the plant cells with the recombinant vector of the present invention, wherein said transformation can be mediated by Agrobacterium tumefaciens. Moreover, the method of the present invention comprises a step of amplifying the resulting transgenic organism in the media containing toxoflavin. The transgenic organism can survive in the media containing toxoflavin while the non-transgenic organism cannot survive in the media containing toxoflavin. As a result, transgenic plants can be easily selected.

For achieving still another object of the present invention, a method of producing transgenic plants comprising the following steps is provided:

carrying out the transformation of plant cells with the recombinant vector of the present invention;

amplifying the resulting transformed plant cells in the media containing toxoflavin; and

redifferentiating the transgenic plants from thus obtained transformed plant cells. The method of the present invention comprises a step of carrying out the transformation of plant cells with the recombinant vector of the present invention, wherein said transformation can be mediated by Agrobacterium tumefaciens. Moreover, the method of the present invention comprises a step of amplifying the resulting transformed plant cells in the media containing toxoflavin. The transgenic organism can survive in the media containing toxoflavin while non-transgenic organism cannot survive in the media containing toxoflavin. Additionally, the method of the present invention comprises a step of redifferentiating the transgenic plants from thus obtained transformed plant cells. Method of redifferentiating the transgenic plants from the obtained transformed plant cells can be any method publicly known in the pertinent art.

The following examples describe the present invention in detail and are illustrative rather than limiting. It should be understood that there may be other embodiments which fall within the spirit and scope of the invention and the scope of the present invention is not limited to the examples.

### EXAMPLES

### Experimental Example 1

### Condition for Culturing Bacterial Cells

Paenibacillus polymyxa cells were cultured in liquid or solid LB medium at 28□. All of Escherichia coli cells were cultured in liquid or solid LB medium at 37□. Antibiotics were used with the following concentration: rifampicin 50 μg/m1; tetracycline 10 μg/ml, kanamycin 30 μg/ml; ampicillin 100 μg/ml; chloramphenicol 25 μg/ml.

### Experimental Example 2

### Enzyme Treatment of DNA

### 1. Preparation of Chromosomal DNA

Extraction of chromosomal DNA from Paenibacillus polymyxa was carried out with a modified lysozyme-sodium dodecyl sulfate (SDS) dissolution method (Leach, J. E. et al., 1990. MPMI. 3:238-246). Bacteria were cultured in LB medium (500 ml) comprising an appropriate antibiotic at 230 rpm, 28□. Bacterial cells were collected via centrifuge. Bacterial pellets were washed with 1 ml of 0.9% NaCl solution, and dissolved in 330 μl GTE solution (50 mM glucose, 25 mM Tris-HCl [pH 8.0], 10 mM EDTA [pH 8.0]). Subsequently, 3 μl lysozyme (50 mg/ml) was added and the reaction was carried out for 30 min at 37□. The cells were then disrupted using 17 μl of 10% SDS and the reaction was carried out at 37□ for 10 min. 10 μl of RNaseA (10 mg/ml) was added and the reaction was carried out at 37□ for 1 hr. 17 μl of 0.5M EDTA was added and the reaction was carried out at 37□ for 10 min. 2.5 μl of proteinase K (20 mg/ml) solution was added and the reaction was carried out at 37□ for 6 hrs. A mixture of phenol:chloroform:isoamyl alcohol in ratio of 25:24:1 (v:v:v) was added and the resulting mixture was vigorously stirred for 5 min. After centrifuge at 16,816×g for 5 min, supernatant was transferred to a new tube to which phenol was added and the extraction was carried out twice. A mixture of chloroform: isoamyl alcohol in ratio of 24:1 (v:v) was added to the tube, with the volume same as that of said supernatant. One volume of 3M sodium acetate [pH 7.0] and 2 volume of 95% ethanol were added. The mixture was centrifuged at 16,816×g for 15 min. After the centrifuge, the supernatant was carefully decanted and the pellet was washed with 70% ethanol. After all the ethanol was evaporated, the pellet was dissolved in 0.2 ml TE [pH 8.0] and kept at −20□.

### 2. Preparation of Plasmid DNA

E. coli plasmid DNA was prepared using an alkaline lysis method (Sambrook, J. et.al., 1989. Molecular Cloning: A Laboratory Manula. 2nd ed. Cold Spring Harbor Laboratory, Cold Sprong Harbor, N.Y.). E. coli cells were cultured in 2 mL LB media comprising appropriate antibiotics at 200 rpm, 37□. Bacterial cells were collected via centrifuge at 16,816×g for 1 min. Supernatant was removed and the bacterial pellets were admixed well with 100 μl ice-cold solution I (50 mM glucose, 25 mM Tris-HCl [pH 8.0], 10 mM EDTA [pH 8.0]), and then 5 μl of RNaseA solution (20 μg/ml) was added. 200 μl of solution II (0.2N NaOH, 1% SDS) was added and the resulting mixture was gently shaken. 150 μof ice-cold solution III (5M potassium acetate 60 ml, glacial acetic acid 11.5 ml, sterile distilled water 28.5 ml) was added and mixed well. Bacterial lysate was centrifuged at 16,816×g, 4□, for 10 min. After subsequent treatment with phenol, only the supernatant was transferred to a new tube to which 1 ml of 95% ethanol was added and admixed well. DNA pellet was obtained via centrifuge at 16,816×g, 4□, for 15 min. The pellet was washed with 70% ethanol and dissolved in 30 μl TE [pH 8.0] and kept at 4□.

3. Agarose Gel Electrophoresis with an Enzyme

Restriction enzymes, calf intestinal alkaline phosphatase, T4 DNA ligase and other relating agents were purchased from Takara (Japan), Boehringer Mannheim (Mannheim, Germany), Stratagene (La Jolla, Calif.), Gibco BLR (Gaithersburg, Md.) and Sigma (St. Louis, Mo.). Analysis conditions were followed as described in manufacturer's instructions. Bacterial DNA was digested with various endonucleases and then separated using 0.7% (w/v) agarose gel (Sigma) with 0.5×TBE buffer system (45 mM Tris-borate, 1 mM EDTA) (Ausubel, F. M. 1991. Current Protocols in Molecular Biology. Wiley Interscience. New York). Specifically, DNA was mixed well and loaded to the gel using gel-loading buffer (comprising 0.25% bromophenol blue, 0.25% xylene cyanol FF, 15% ficoll in water). The resulting gel was stained for 30 min in 0.5 μg/ml ethidium bromide solution and then observed under a transilluminator.

4. Isolation of DNA Fragment from Agarose Gel

DNA fragment was isolated from an agarose gel using QIAEX II gel extraction kit (150) (QIAGEN, Germany).

### Experimental Example 3

### Transformation using Calcium Chloride

As described by Maniatis et al. (Maniatis, T. et al., 1982. Molecular Cloning; A Laboratory Manual, Cold Spring Harbor Lab Press, New York), E. coli transformation was carried out using calcium chloride. To prepare competent cells, E. coli cells were cultured for 12 hrs and further cultured at 230 rpm, 37□ till the exponential phase was reached (A600=0.6). The medium was then collected and kept on ice for 20 min. The pellet was obtained from the medium via centrifuge at 4□, 2,700×g. CaCl₂ solution (sterilized 10 mM calcium chloride and 10% glycerol) kept on ice was added and mixed well. After the reaction on ice for 20 min, the mixture was centrifuged at 2,700×g, 4□ for 10 min. To the pellet, CaCl₂ solution kept on ice was added and mixed well. The mixture was aliquoted (0.1 ml each) to a pre-chilled centrifuge tube and kept at −70□ until further use. For transformation, 85 μl of TE buffer was added to 15 μl of ligation mixture. To the competent cells that have been slowly thawed on ice, said pre-chilled ligation mixture was added carefully and admixed well. The reaction was carried out for 20 min. After subjected to a heat shock treatment at 42□ in 1 ml LB, the cells were cultured for 1 hr at 37□ without shaking. The resulting cells were evenly plated on solid LB media comprising antibiotics.

### Experimental Example 4

### Isolation of Toxoflavin-Degrading Bacteria

2 mL of minimal medium was added to 1 mg of field soil sample. After culturing at 37□ for 48 hrs, it was evenly plated on LB agar media comprising toxoflavin in concentration of 40 μg/ml. After culturing for 1 to 2 days, a single colony was separated. Rice seeds were sterilized and cultured in minimal medium at 37□ for 24 hrs. Then, 40 μg/ml of toxoflavin was added and cultured again for 48 hrs. The resulting culture was plated evenly on LB agar medium comprising 40 μg/ml of toxoflavin. After culturing for 2 to 3 days, single colonies were separated and again plated evenly on LB agar medium to obtain pure single colonies.

### Experimental Example 5

### Characterization of Bacterial Cells

In order to characterize the isolated bacterial cells, their physiological and culturing characteristics were analyzed by Biolog program analysis, GC-FAME (gas chromatography of fatty acid methyl esters), and 16S rDNA sequence analysis.

Carbon source utilization profiles of the isolated cells were carried out three times in accordance with the manufacture's instruction by Biolog microplates (Biolog GN MicroPlate; Biolog, Hayward, Calif.). After culturing for 24 hrs and 48 hrs, respectively, plates were read using MicroLog 3-Automated Microstation system (Biolog). With reference to Microlog Gram-positive database (Version 4.0), the bacteria were characterized.

For an analysis of fatty acid methyl ester, nine expected types of bacterial cells which have been isolated above were cultured in Trypticase soy broth (Becton Dickinson and Co., Franklin Lakes, N.J.) agar plate for 48 hrs at 28□. Fatty acid methyl esters were extracted based on a standard method (Sasser, M. 1997. Identification of bacteria by gas chromatography of cellular fatty acids. Technical note #101. MDMI, Newark, Del.). Fatty acids were analyzed using Sherlock Microbial Identification System Version 2.11 (MIDI Inc., Newark, Del.). Analysis of the isolated fatty acid methyl ester was carried out three times.

16S rDNA sequencing of the isolated bacterial cells were carried out by PCR with total reaction volume of 50 μl comprising 5 μl of 10×PCR buffer (Takara Bio Inc., Otsu, Japan), 5 μl of each of dNTP (2.5 mM, Takara), 1 μl of primer (100 pmol, 27 mF: 5′AGAGTTTGATCMTGGCTCAG3′ (SEQ ID NO: 3), 1492 mR: 5′GGYTACCTTGTTACGACTT-3′ (SEQ ID NO: 4)), 0.5 μl of Taq polymerase (250 U/μl, Takara) and 2 μl of bacterial floating substances (A_{600 nm}=0.1). PCR amplification was performed with an automated thermal cycler (model PTC-150, Perkin-Elmer Cetus, Norwalk, Conn.). First denaturing condition was 5 min at 94□, and the reaction was carried out 29 times with the following condition; denaturation 94□/1 min, annealing 55□/1 min, elongation 72□/1.5 min (at the very end an amplification step was added once; 72□/10 min).

Amplified DNA was cloned at SmaI site of pBluescript II (SK+) (Stratagene, Cedat Creek, Tex.) using a method described by Sambrook et al. (Sambrook, J. et al., 1989. Molecular Cloning: A Laboratory Manula. 2nd ed. Cold Spring Harbor Laboratory, Cold Sprong Harbor, N.Y.).

DNA sequencing AB13700 automated DNA sequencer (Applied Biosystems Ins., Foster City, Calif.) was employed for DNA sequencing. Results obtained from DNA sequencing was analyzed with BLAST Program of National Center for Biotechnology Institute (Altschul, S. F. et al., 1990. Basic local alignment search tool. J. Mol. Biol. 215:403-410).

### Experimental Example 6

### Construction of P. polymyxa JH2 Cosmid Library

Chromosomal DNA was prepared from 500 ml of P. polymyxa JH2 culture and then partially digested with Sau3A. Fragments with length of 20˜30 kb were separated by sucrose gradient centrifugation (10 to 40% [wt/vol]) at 24,000 rpm, room temperature for 24 hrs. Subsequently, they are ligated with pLAFR3 (Tra⁻, Mob⁺, RK2 replicon, Tet^{r}, Staskawicz, B. et al., 1987. Molecular characterization of cloned avirulence genes from race 0 and race 1 of Pseudomonas syringar pv. glycinea. J. Bacteriol 169:5789-5794).

Ligated DNA was wrapped by bacteriophage λ in accordance with the manufacturer's instruction (Promega, Madison, USA). E. coli HB 101(F⁻ mcrB mrr hsdS20 (r_{B}⁻m_{B}⁻) recA13 leuB6 ara-14 proA2 lacY1 galK2 xyl-5 mtl-1 rpsL20 (Sₘ^{R}) suoE44 λ⁻, Gibco BRL) was transformed with said bacteriophage λ.

### Experimental Example 7

### Library Screening

Library was screened by culturing the cells in LB liquid media at 230 rpm, 37□. After culturing the cells in liquid LB media comprising 40 μg/ml of toxoflavin for 3 hrs, they were cultured for additional 12 hrs. Liquid media was spread evenly onto the solid LB media to which 40 μg/ml of toxoflavin was added. For this solid media, colonies were observed one day after culturing at 37□.

### Experimental Example 8

### Sequencing

1. Sequencing Reaction

Template plasmid DNA was used in accordance with the manufacture's instructions of QIAprep Spin Miniprep kit (QIAGEN, Germany). Reagents comprising 1 μg of BigDye terminat or ready reaction mix, 2 pmol T7 promoter primer, template plasmid DNA 5 μl (100-200 ng) were admixed well. The reactants were transferred to 0.2 ml PCR tube, heated at 95□ for 5 min and subjected to an amplification process using a thermal cycler (Minicycler™ PTC-150 (MJ Research, Watertown, Mass.)) with 25 cycles of the following reaction condition; 20 sec, 95□/10 sec, 55□/4 min, 60□.

2. Purification of PCR Products

When a sequencing reaction is over, PCT product (10 μl) is transferred to a 1.5 ml centrifuge tube. 17 reagents (distilled water 26 μl and 95% ethanol 64 μl) were added to 10 μl of the reaction product. After mixing well, it is kept at room temperature for 15 min.

Centrifuge is carried out at 16,816×g, room temperature for 20 min. Supernatant is discarded and pellet is washed with 250 μl of 70% ethanol and dried in air. Final product is kept at the temperature of −20□.

3. DNA Sequencing and Data Analysis

pJ9 (plasmid wherein the cosmid library is cloned) insertion DNA is digested with an appropriate restriction enzyme and subcloned into pBluescript II SK(+) before sequencing. Universal primer (SEQ ID NO: 5) and reverse primers (SEQ ID NO: 6) are used for a basic reaction while a synthetic primer (SEQ ID NO: 7 and SEQ ID NO: 8) was used for sequencing of complete double strands. DNA sequencing data was analyzed using BLAST program (Gish, W. et al., 1993. Identification of protein coding regions by database similarity search. Nat. Genet. 3:266-272), MEGALIGN Software (DNASTAR) and GENETYX-WIN Software (Software Development, Tokyo, Japan).

### Experimental Example 9

### Overexpression and Partial Purification of His-TflA

tflA gene of P. polymyxa JH2 was amplified via PCR using primers having sequences of SEQ ID NO: 9 or SEQ ID NO: 10, and cloned into pET14b vector (Novagen, Madison, Wis., USA) using NdeI/BamHI. E. coli BL21 (DE3) (pLysS) bacterial cells to which pET14b has been incorporated were cultured in liquid LB media comprising ampicillin and chloramphenicol, while shaking at 37□. When OD_{600 nm} reached 0.8, IPTG was added to the media to obtain its final concentration of 1 mM. After culturing at 37□ for 2 hrs, the cells were collected by centrifuge. 50 mM sodium phosphate (pH 6.5) was added and mixed well with pellet, and the mixture was sonicated. Concentration of partially purified proteins which were obtained by the centrifuge were determined with Bradford method using BSA as a standard (Bradford, M. M. 1976. A rapid and sensitive method for the quantitation of microgram quantities of protein utilizing the principle of protein-dye binding. Anal. Biochem. 72:248-254). The proteins were analyzed by SDS-PAGE and staining by Coomassie Blue.

### Experimental Example 10

### Purification of N-Terminal His-TflA

For better purification, N-terminal His-tagged TflA was used. E. coli BL21 (DE3) (pLysS) carrying pET14b vector (Novagen, Madison, Wis., USA) wherein tflA has been cloned was cultured in LB liquid media. IPTG was used to induce overexpression of proteins. After harvesting, the cells were disrupted by sonication in the presence of 50 mM sodium phosphate (ph 6.5), and centrifuged at 10,000×g, 4□ for 20 min. Supernatant was loaded onto the top of Ni-NTA spin column (QIAGEN, Valencia, Calif., USA). His-tagged protein was centrifuged at 1,000×g, 4□ for 2 min and then attached to Ni-NTA matrix. By washing the matrix twice with washing buffer (20 mM imidazole, 50 mM sodium phosphate (pH 6.5)), unbound proteins were removed. By applying 0.1 ml elution buffer 1 (10 mM imidazole, 50 mM sodium phosphate (pH 6.5)) and 0.1 ml elution buffer 2 (10 mM imidazole, 50 mM sodium phosphate (pH 6.5)) in order, His-tagged proteins were dissolved and eluted. Thus obtained proteins were dialyzed against 50 mM sodium phosphate (pH 6.5) to remove imidazole compounds. Concentration of the purified proteins was determined with Bradford method with standards (Bradford, M. M. 1976. A rapid and sensitive method for the quantitation of microgram quantities of protein utilizing the principle of protein-dye binding. Anal. Biochem. 72:248-254).

### Experimental Example 11

### Enzyme Characteristics of His-TflA

1. Enzyme Analysis

TflA activity was determined using thin layer chromatography (TLC) plate, which basically follows a change in products from the reaction between toxoflavin and enzyme reaction mixture. 5 μM TflA protein, 10 μM MnCl₂ and 5 mM DTT (dithiothreitol) were added to analysis buffer (50 mM sodium phosphate (pH 6.5)) to give 200 μl mixture and the reaction was carried out at 25□. Concentration of toxoflavin was adjusted to be 100 μM just before starting the reaction. After 10 min of the reaction, 200 μl of chloroform was added to stop the reaction. The chloroform layer was dried completely, then 10 μl of 100% methanol was added. An aliquot taken from the reaction mixture comprising methanol was applied on a TLC plate using pipette. The resulting plate was placed in TLC chamber containing mixed solvents of chloroform and methanol (95:5, V:V) at room temperature. TLC was visualized under UV illuminator (254 nm and 365 nm).

2. Metal Ion Effect on the Enzyme Activity

Effect of metal ions on the enzyme activity was investigated. All metal ions were used with 1 mM concentration for the investigation.

3. Effect of pH and Temperature on the Enzyme Activity

Under various pH condition, tflA activity was measured using 50 mM sodium phosphate buffer (pH 4.0-8.0) at 25□. Enzyme activity was measured in the temperature range of 10-40□ using 50 mM sodium phosphate buffer (pH 6.5).

4. Enzyme Kinetics

Kinetic parameters (Km and Vmax) are determined from Lineweaver-Burk plot which is established with data obtained from different concentration of toxoflavin (80-200 μM). Each of experimental points is a mean value that is calculated from data obtained from at least of three measurements.

### Example 1

### Isolation of Toxoflavin-Degrading Gene

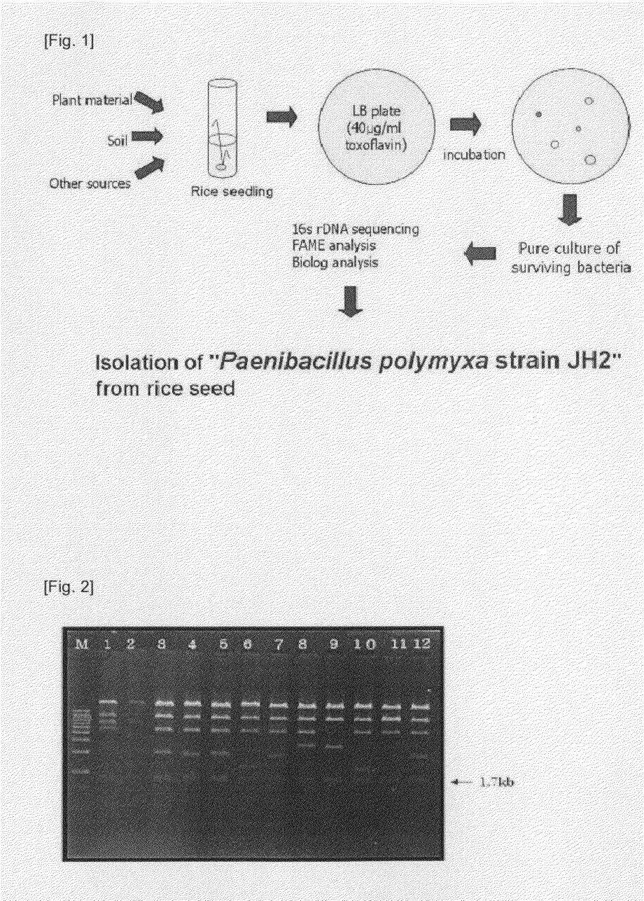

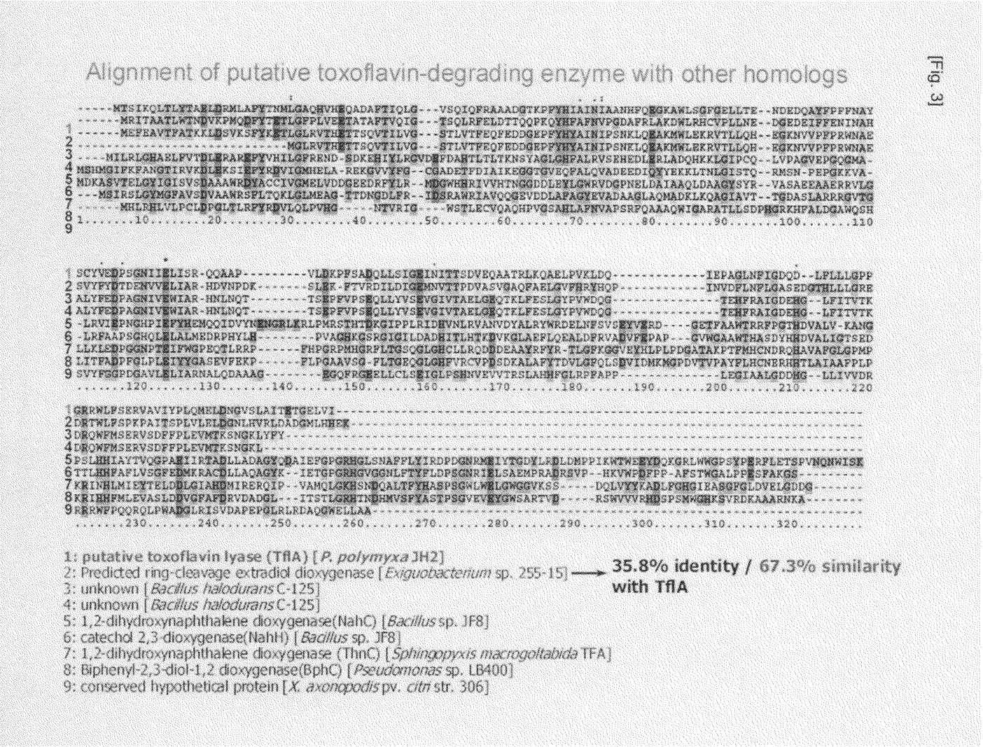

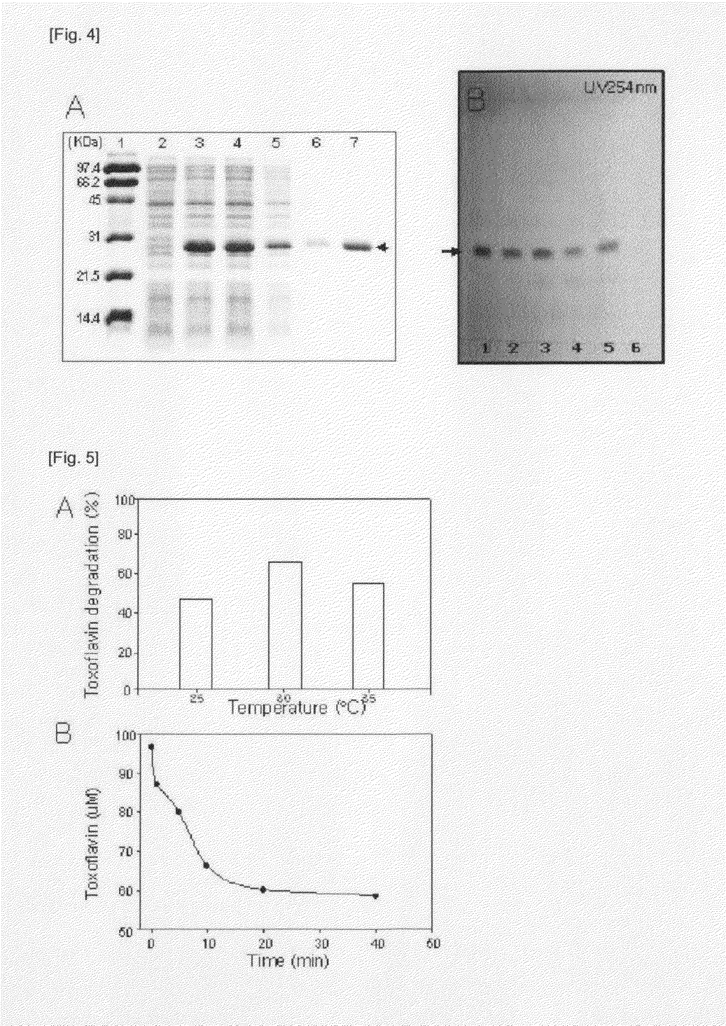

500 different kinds of bacteria were obtained from mountain or field soil, rice paddy soil, and rice seeds, etc., and they were cultured in minimal medium to which toxoflavin was added. As a result, the bacterial cells which were able to grow after degrading toxoflavin were separated. Among the bacteria isolated from rice seeds, the bacterial cells that can specifically degrade toxoflavin were separated to their pure state. They were characterized by 16s DNA sequencing analysis, fatty acid analysis and Biolog analysis. As a result, the cells were identified as Paenibacillus polymyxa and named JH2 (see, FIG. 1). Genomic DNA library from P. polymyxa JH2 was constructed in E. coli HB 101. From the constructed library clones, a colony which can survive in media comprising toxin was selected. A clone which has been prepared by digestion with restriction enzymes of said isolated DNA clone was used to confirm the minimal DNA fragment that is required for degrading toxoflavin (see, FIG. 2). 1.5 kb long EcoRI fragment, which is a minimal clone for degrading the toxin, was subjected to DNA sequencing analysis to confirm ORF having 666 bp (SEQ ID NO: 1). From NCBI BLAST analysis, it was found that it has a similarity of 67.3% with ring-cleavage extradiol dioxygenase of Exiguobacterium sp. (see, FIG. 3). It was found that tflA gene which appears to be involved with the degradation of toxoflavin is a new useful gene that has not been reported before.

### Example 2

### Expression of Toxin-Degrading tflA Gene and Degradation of the Toxin by Purified Proteins

pET14-b (T7 promoter expression vector, Amp^{r}, Novagen) was used to express tflA gene which can degrade toxoflavin. Said gene which has been amplified using PCR was cloned into pBluescript II SK(+)(ColEI, MCS-lacZα, Amp^{r} cloning vector, Ampr, Stratagene) and its sequence was analyzed. Clones having no problem with PCR were cloned again into pET14-b (i.e., pH904). E. coli BL21(DE3/pLysS) was transformed with said pH904 and induced by addition of IPTG (1 mM). His-TflA protein (26.7 kDa) was then purified using Ni-column (see, FIG. 4A). Thus purified TflA protein was tested for its ability of degrading toxoflavin, and it was found that degradation of toxoflavin requires the co-presence of DTT and Mn²⁺ (see, FIG. 4B).

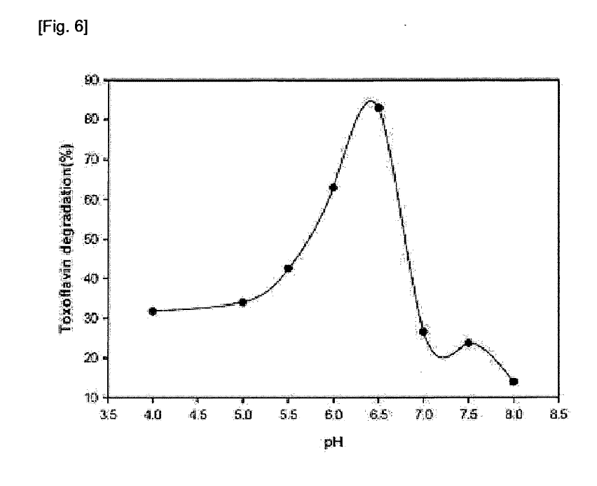

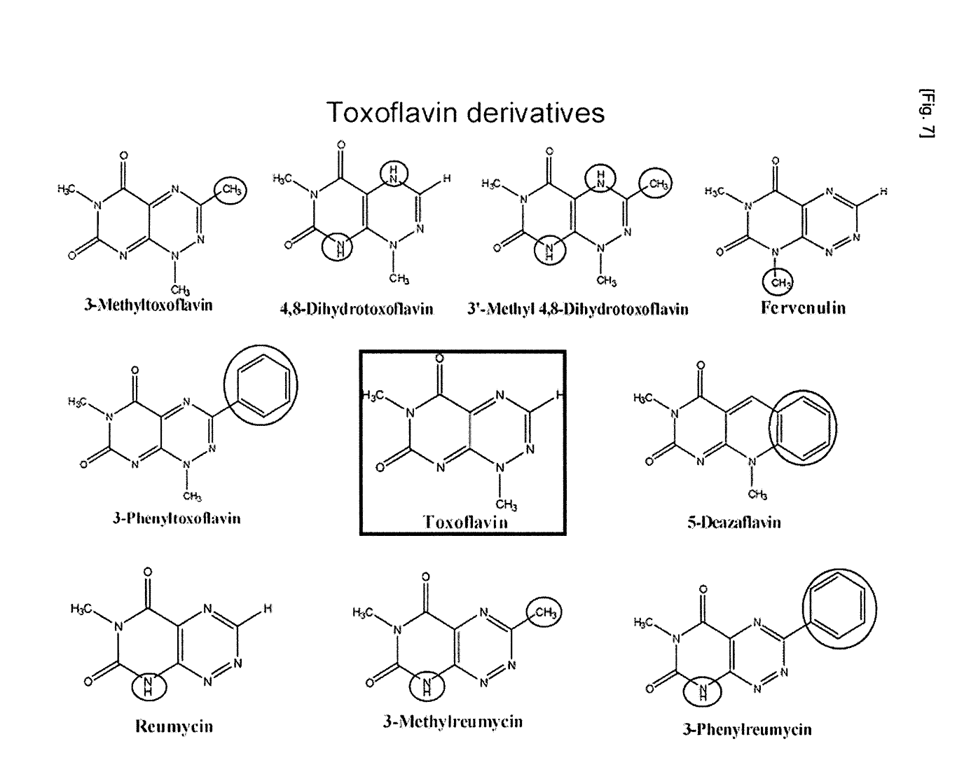

In order to obtain temperature profile of toxoflavin degradation by TflA protein, the amount of toxoflavin which has been degraded by the protein was determined at various temperatures of 20, 25, 30, 35 and 40□. Consequently, it was found that TflA protein shows its highest activity of degradation at 30□ (see, FIG. 5A), and the optimum pH was pH 6.5 (see, FIG. 6). To measure specific activity of TflA protein, degradation of toxoflavin was checked every 10 min. It was found that the specific activity of TflA protein is 0.0413 μmoles/min/mg (see, FIG. 5B).

### Example 3

### Degradation of Toxoflavin and its Derivatives by TflA Protein

1) Synthesis of Toxoflavin and its Derivatives

For carrying out a study about the reaction mechanism for toxin degradation by TflA protein, the inventors of the present invention obtained toxoflavin and its derivatives from Dr. Tomohisa Nagamatsu, a professor of Okayama University JAPAN, who is a co-worker of this project (see, FIG. 7). Derivatives of toxoflavin including, 3-methyltoxoflavin having a methyl group at carbon number 3 (for carbon and nitrogen numbering, original unmodified toxoflavin was taken as a standard compound unless stated otherwise), 4,8-dihydrotoxoflavin having hydrogen atoms at nitrogen number 4 and 8, 3′-methyl 4,8-dihydrotoxoflavin having a methyl group at carbon number 3 and hydrogen atoms at nitrogen number 4 and 8, fervenulin having a methyl group at nitrogen number 8 instead of nitrogen number 1, 3-phenyltoxoflavin having a phenyl group at carbon number 3, 5-deazaflavin having an additional ring structure, reumycin not having a methyl group at nitrogen number 1, 3-methylreumycin having a methyl group at carbon number 3 of reumycin, and 3-phenylreumycin having a phenyl group at carbon number reumycin, were used for determination of degradation by TflA protein.

2) Degradation of Toxoflavin and its Derivatives by TflA

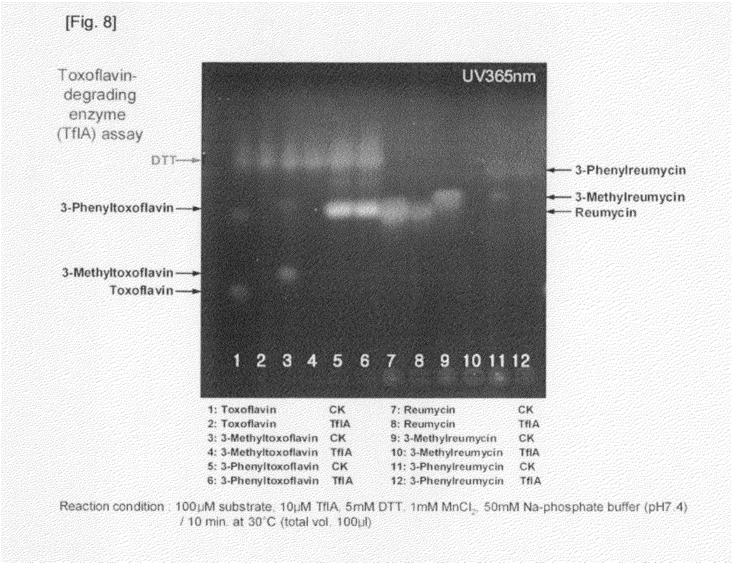

After conducting a serial test to determine the degradation of toxoflavin and its derivatives by TflA protein, it was found that, toxoflavin, 3-methyltoxoflavin, 4,8-dihydrotoxoflavin, 3-methylreumycin were all completely degraded within given time. However, 3-phenyltoxoflavin, 3-phenylreumycin, and 5-deazaflavin were not degraded at all and reumycin, 3-methyl 4,8-dihydrotoxoflavin, and fervenulin were partially degraded. Taken all together, because the derivatives having a phenyl group at carbon number 3 did not undergo any degradation, it is believed that TflA protein may recognize the specific ring structure around carbon number 3 of toxoflavin (see, FIG. 8).

### Example 4

### Kinetics of TflA Protein

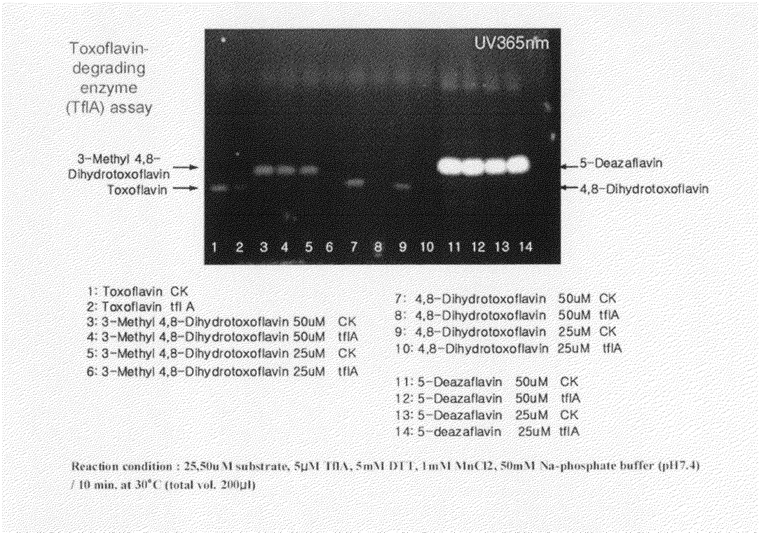

Michaelis constant (Kₘ), maximum reaction rate (Vₘₐₓ), and specific activity were determined for His-TflA protein which can degrade toxoflavin. Activity of degrading toxoflavin toxin was confirmed by TLC analysis. As a result, it was found Kₘ and Vₘₐₓ value for His-TflA protein was 69.72 μM and −0.45 U/mg, respectively. Specific activity was found to be 0.4 μmol/mg (see, FIG. 9).

### Example 5

### Preparation of Transgenic Plants

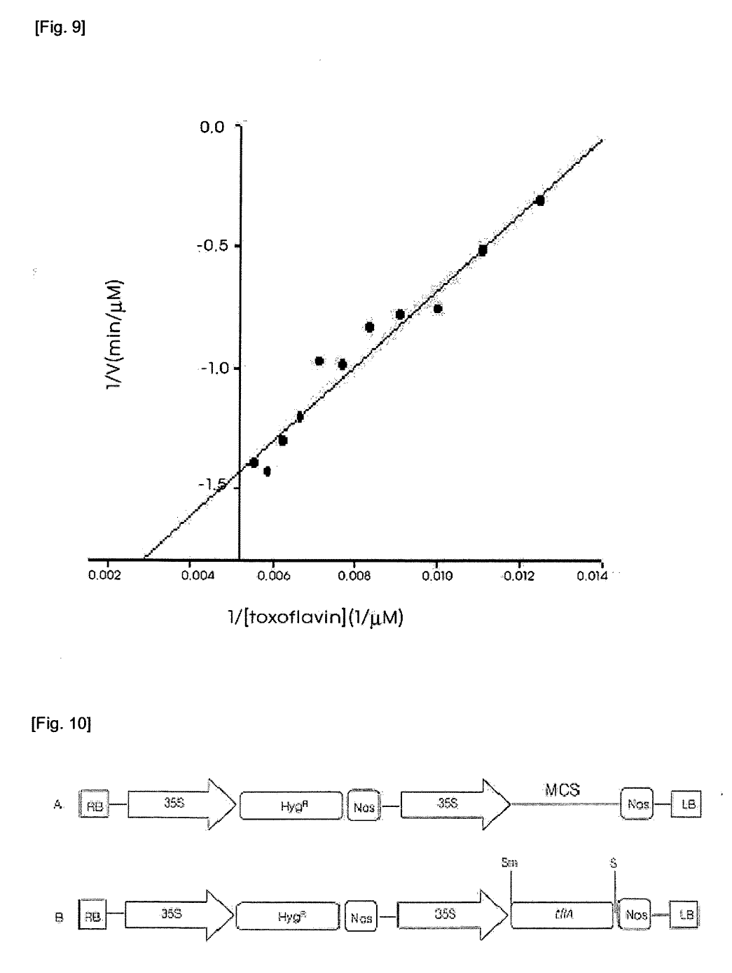

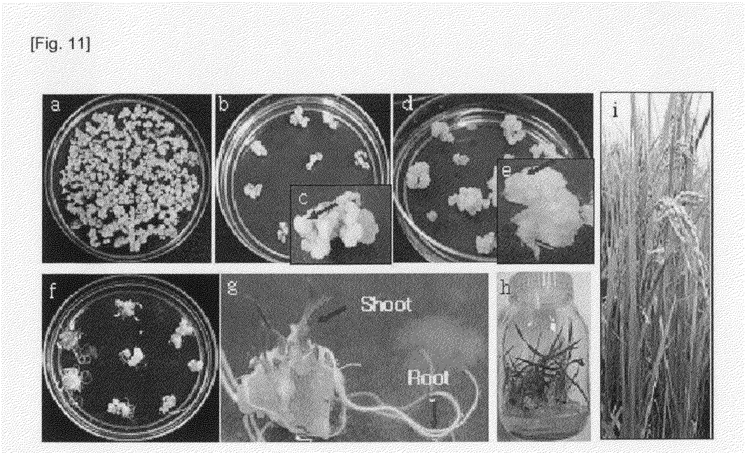

The vector which has been used for transformation was pCamLA wherein hygromycin phosphotransferase, Hyg^{R} is comprised inside T-DNA and kanamycin resistant gene is comprised outside T-DNA (see, FIG. 10). Bacterial cells used for the transformation was Agrobacterium tumefaciens LBA4404. Agrobacterium was cultured in AB minimal medium. The cultured cells were recovered and diluted with AA liquid medium comprising acetosyringone (3,5-dimethoxy-4-hydroxy acetophenone, Aldrich). Rice callus obtained from varieties including Donjinbyeo, Chucheongbyeo, and Nipponbare was immersed for 3 to 5 min in said cell mixture. The resulting callus was dried using a sterilized filter paper and placed in the media to co-cultivate with Agrobacterium under dark condition at 28□ for 3 days. Agrobacterium was removed from the callus that has been cultured for 3 days, and the resulting callus was placed in N6 selection medium. By culturing under light condition at 25□ for about 30 days, only the proliferated callus was selected, which was then placed again in the media for redifferentiation. After obtaining the plants that have been redifferentiated, they were transferred to a media devoid of any substance for controlling growth so that root growth can freely occur (see, FIG. 11). Individual plants which have normally grown shoots and roots in the media comprising hygromycin were selected and acclimated. Thus obtained transgenic plants were transplanted in a pot and kept in a greenhouse. From the transgenic plants that have been obtained from repeating experiments, plants that appear to have undergone normal growing process judged from their appearance were selected. Consequently, T1 and T2 transgenic rice plants were established therefrom as summarized in Table 1.

TABLE 1
List of transgenic rice plant lines

| Rice cultivar | Gene | No. of Line | Present |
|---|---|---|---|
| Donjinbyeo | tflA | Dt 46 | T1 plant |
| Nipponbare | tflA | Nt 41 | T1 plant |
| Chucheongbyeo | tflA | Ct 43 | T1 plant |

### Example 6

### Analysis of Transgenic Plants (T2 Plant)

1) Southern Blot Analysis

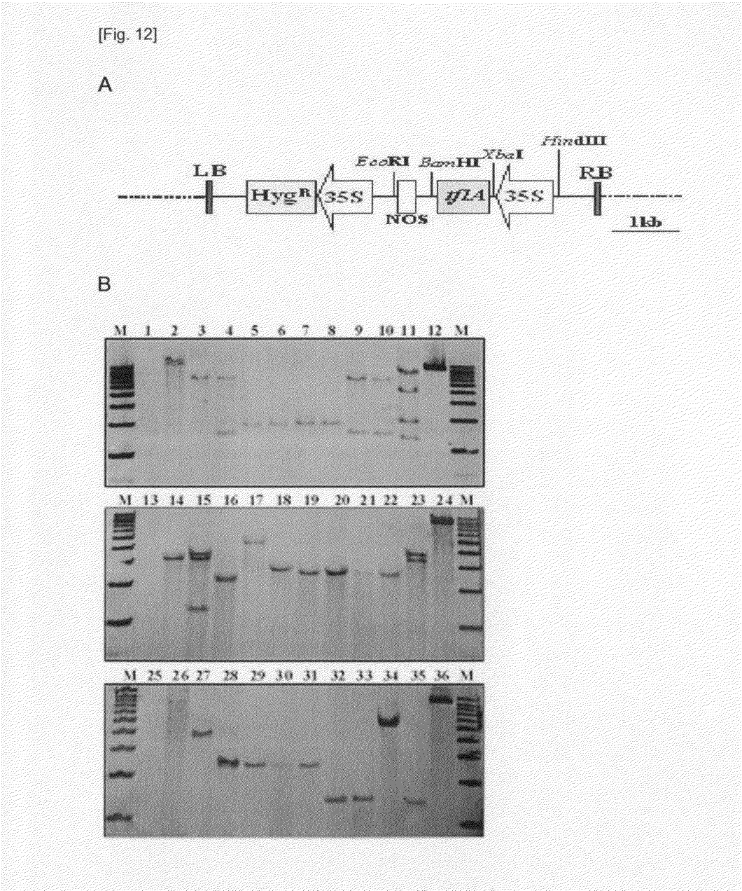

Genomic DNA was extracted from one gram leaf tissue of transgenic rice plant using a standard method. Genomic DNA (15-20 μg) was treated with the restriction enzyme EcoRI and separated in an agarose gel (0.7%). After blotting the separated DNA to a membrane, a hybridization reaction was carried out using a probe. The probe used was tflA fragment (2.5 kb) which comprises 3′ NOS terminator. The result of Southern blot analysis obtained from the hybridization between leaf tissue DNA of the transgenic plant at T2 generation and the OA probe is shown in FIG. 12B and Table 2. For both rice cultivars of Donjinbyeo and Chucheongbyeo, various integration pattern and copy number were found.

2) Western Blot Analysis

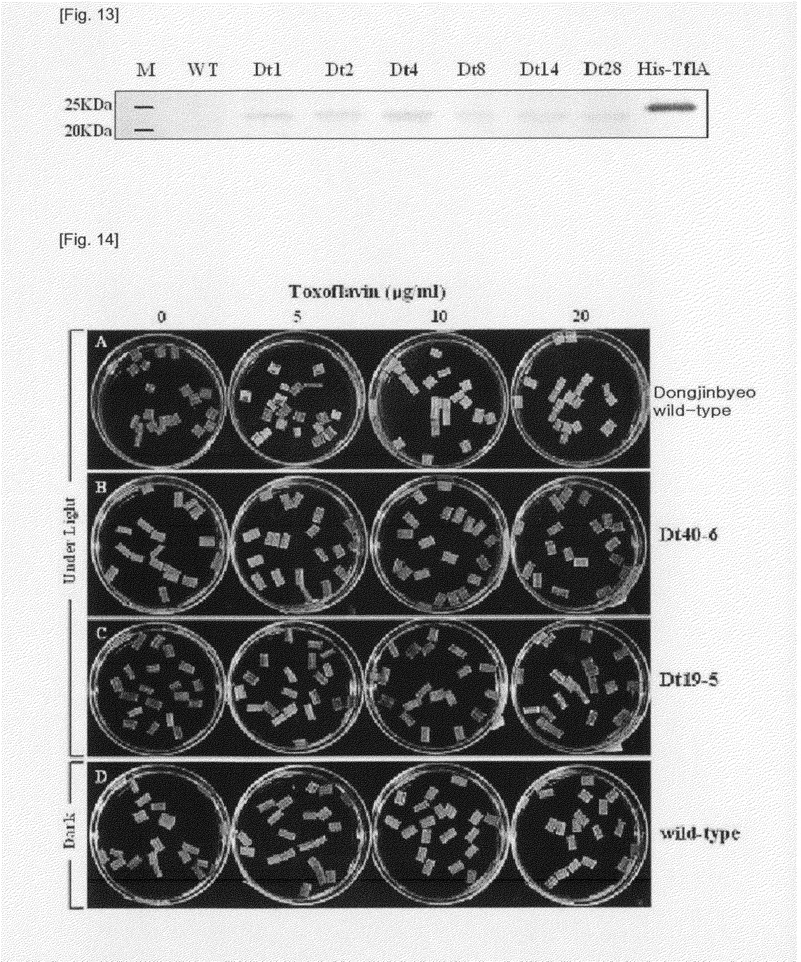

Leaf tissue from the transgenic rice plant at T2 generation (300 mg) was collected and crushed using liquid nitrogen. Crushing buffer (50 mM Tris-HCl pH7.5, 150 mM NaCl, 1 mM EDTA, 10% glycerol, 1 mM PMSF, 0.05% Tween 20, protease inhibitor cocktail) was added thereto and the mixture was centrifuged for 15 min (4□, 15,000 rpm). Supernatant was taken and centrifuged again to extract total soluble protein. To 2× sample butter, the total soluble protein (15 μl) was added and the resulting mixture was heated in hot boiling water of 100□ for 5 min. The proteins were then subjected to SDS-PAGE and subsequently blotted to a PVDF membrane. After treating the membrane with a blocking solution (5% skim milk in TBS-T), it was treated with anti-TflA antibody and immunoPure® Antibody. NBT/BCIP Detection Kit (Amersham, England) was employed for signal detection. As it is shown in FIG. 13, TflA protein was normally expressed in the transgenic rice plant at T2 generation.

### Example 7

### Selection of the Transgenic Rice Plant (T3 Plant)

Plants which have normally grown shoots and roots in the media comprising hygromycin were selected and acclimated. Thus obtained transgenic plants were transplanted to a pot and kept in a greenhouse. From the transgenic plants that have been obtained from repeating experiments, plants that appear to have undergone normal growing process judged from their appearance were selected. Rice seeds (T1 and T2) were taken from said plants and used for a determination of resistance to hygromycin. Testa was removed from mature seeds, and the resulting seeds were immersed in 100% ethanol for 1 min. Surface sterilization of the seeds was performed using 2% sodium hypochloride solution for 20 min while stirring. Thus sterilized seeds were washed with sterile water three times and placed in 1/2 MS media to which hygromycin has been added (50 mg/L). Cultivation was carried out under continuous light (3000 lux) at the temperature of 26□. 10 days after the planting, its resistance to hygromycin was determined by observing the growth of stems and roots of the rice plant. From the seeds taken from each of the rice cultivar, separation ratio for the genes inducing a resistance to hygromycin was determined. As shown in Table 2 in the following, transgenic T3 rice plant of two cultivars was established and used for further study.

TABLE 2
List of transgenic rice plants

| Gene | Rice cultivar | T1 lines | T2 lines | T3 lines |
|---|---|---|---|---|
| tflA | Dongjinbyeo | 25 | 21 | 16 |
| | Chucheongbyeo | 15 | 12 | 8 |

### Example 8

### Determination of Phenotype of Transgenic Plant (T3 Plant)

Resistance to toxoflavin was tested for the leaves taken from the transgenic T3 rice plant which had grown from 4 to 5 leaves. A preliminary investigation revealed that the activation of toxoflavin requires light. Thus, the experiment of the present invention was carried out under light. To a Petri dish having a size of 60×15 mm, 5 ml of sterilized water was added and toxoflavin was also added in various concentrations of 0, 25, 50 or 100 μM. Leaves which have been taken from the transgenic T3 rice plant grown from 4 to 5 leaves and then cut in the size of 3×4 mm were subjected to the treatment with toxoflavin for 48 hrs, in which the treatment includes a growing phase with 16 hrs, 25□ under light and 8 hrs, 25□ under dark. As it is depicted in FIG. 14, 40 hrs after the treatment with toxoflavin discoloration due to the toxin started to show up for the leaves of wild rice. However, for the leaves of the transgenic plants to which tflA gene has been introduced, no such discoloration was observed after the toxoflavin treatment.

### Example 9

### Preparation of a Transformation Vector

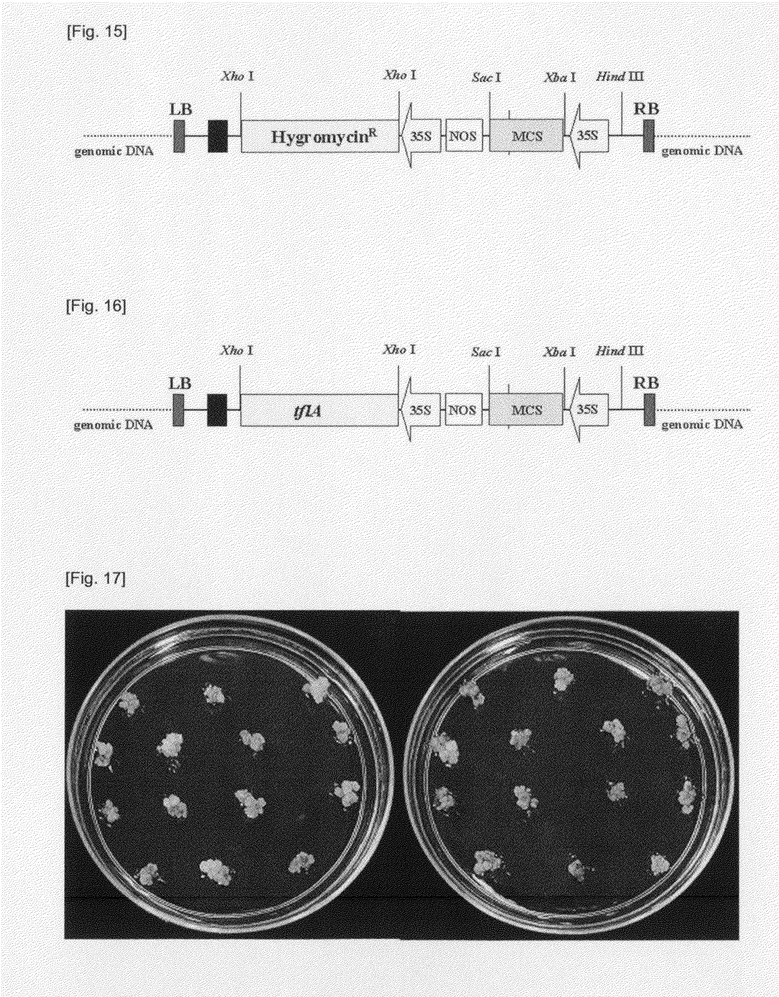

pCamLA and pTflA are the vectors that have been used in the present invention for the transformation. pCamLA carries hygromycin phosphotransferase gene (Hyg^{R}) inside T-DNA and kanamycin resistant gene outside T-DNA (see, FIG. 15). pTflA carries tflA gene (i.e., a gene which is expressed to produce toxoflavin-degrading enzyme) inside T-DNA, 35S promoter and NOS terminator sequence (see, FIG. 16).

The vector that is used for hygromycin selection was pCamLA vector which is derived from a binary vector pCAMBIA1300. It has hygromycin phosphotransferase gene (Hyg^{R}) inside T-DNA and kanamycin resistant gene outside T-DNA. pCamLA was proliferated in E. coli DH5α as host. Plasmid DNA was extracted from the bacteria. Extracted pCamLA was then transformed into Agrobacterium tumerfaciens LBA4404 using an electroporation method.

pTflA vector, which is used for screening toxoflavin, was prepared from pCamLA in which Hyg^{R} gene was deleted by enzyme digestion with XhoI and tflA gene having XhoI adaptor at its start codon and stop codon sites was substituted into said deletion site.

tflA gene having XhoI adaptor was prepared as follows: a PCR amplification of tflA gene was carried out using pJ90 (1.2 kb HindIII fragment which comprises tflA in pBluscript II SK(+)) as a template DNA and J90XhoI-F (5′-CTCGAGATGACTTCGATTAAACAGCTTAC-3′; SEQ ID NO: 11) and J90XhoI-R (5′-CTCGAGTTAGATCACCAGTTCACC-3′; SEQ ID NO: 12) as a primer, and then amplified PCR product was cloned into the Xhol site of pBluscript II SK(+). Sequencing was carried out and the resultant was named as pJX90-6. DNA fragments obtained from the digestion of pJX90-6 with restriction enzyme XhoI was substituted into pCamLA from which Hyg^{R} has been removed. As a result, pTflA was obtained.

### Example 10

### Transformation of the Rice Plant

1) Induction of Callus

Rice seeds (rice grains) that have been harvested in previous year were used for the experiments of the present invention. Specific steps of inducing rice callus are described below.

1. Only the high quality rice seeds were selected (care should be taken not to hurt an embryo bud)

2. The seeds were placed in Falcon tube and shaken in 100% ethanol for 30 sec.

3. 1/2 Chlorox was added thereto and the mixture was incubated for 20 min in a shaking incubator.

4. The seeds were washed 4 to 5 times with sterilized water.

5. The seeds were transferred to 2N6 free media which can induce the formation of callus, while making sure to have the embryo bud of the seed facing upward.

It was found to be appropriate to have a callus inducing condition set at 28□ for 3 to 4 weeks. Especially, four-week-long induction was the best, and it should be never longer than five weeks. The Petri dish used for the present experiments is the one larger than normal dish (i.e., a Petri dish with the size of 100/20 mm was used).

TABLE 3

| | | | |
|---|---|---|---|
| Sucrose | 30 | g | Showa 1900-3260 |
| Casamino acid | 300 | mg | Difco 0230-01-1 |
| Proline | 2.878 | mg | Sigma |
| CHU | 3.981 | g | Sigma C1416 |
| N6-Vitamin | 1 | ml | |
| 2,4-D (Stock 2 mg/ml) | 1 | ml | Sigma D2999(100 g) |

pH 5.8 adjustment

| | | | |
|---|---|---|---|
| Gellan gum | 2 | g | Kanto 17611-13 |

TABLE 4
Stock solution (X 1000)

Media (mg/100 ml)

| Component | MS | N6 | R2 | B5 |
|---|---|---|---|---|
| Inositol | 10000 | — | — | 10000 |
| Nicotinic Acid | 50 | 50 | — | 100 |
| Pyridoxine-HCl | 50 | 50 | — | 100 |
| Thiamine-HCl | 100 | 100 | 100 | 1000 |

2) Transformation of the callus

(1) Co-Inoculation (Dark condition)

3 days before co-inoculation, the induced callus was placed in new 2N6 media and the culture with Agrobacterium was carried out on next day.

1. Agrobacterium is cultured for 48 hrs.

2. Cells are precipitated by centrifuge at 3000 rpm for 5 min.

3. The cells are diluted in AAM media until OD₆₆₀=0.1.

4. An appropriate amount of the callus is put into a tube and immersed in said AAM media for 30 sec.

5. Supernatant is carefully decanted after 30 sec. Then it is sprayed on a filter paper and dried.

6. While the callus is being dried for 20 to 30 min, a filter paper is placed inside a Petri dish and a small amount of AAM media is applied to wet the paper (i.e., 1-2 ml).

7. Dried callus is placed on top of the filter paper and the Petri dish is wrapped well with an aluminum foil. The dish is incubated at the temperature of 24□ for 3 days.

(2) First Selection (Dark Condition)

1. Media comprising 2N6+hygromycin (a selection marker for plants; 10 mg/L (200 ul-50 mg/ml))+cephatoxin 250 mg/L (1 ml -250 mg/ml) is prepared.

2. Callus which has been cultured for 3 days is placed in a sterilized tube and washed once with sterilized water.

3. The callus is washed with a solution containing sterilized water 50 ml plus cephatoxin 50 ul, three times for 20 min each.

4. The callus is dried after being sprayed onto the filter paper.

5. Resulting callus is placed in the 1^{st} selection media.

6. Wrapped with an aluminum foil, the callus is incubated at 28□. Observation is made for 7 days.

(3) Second Selection (Dark Condition)

1. Media comprising 2N6+hygromycin 30 mg/L (600 ul-50 mg/ml)+cephatoxin 250 mg/L (1 ml -250 mg/ml) is prepared.

2. Dark regions correspond to lack of resistance to the toxin. Thus, excluding the dark regions, only the white regions were transferred to the second selection media.

3. Observation is made for three weeks.

(4) The First Differentiation Media for Three Weeks (Light Condition)

(5) The Second Differentiation Media for Three Weeks (Light Condition)

(6) Bottle 1/2 MS

TABLE 5
AAM media

| | |
|---|---|
| 1 L | 500 ml |

| | | | | |
|---|---|---|---|---|
| MSAA | 100 | ml | 50 | ml |
| MS vitamin | 0.5 | ml | 0.5 | ml |
| Casamino acid | 0.5 | g | 0.25 | g |
| Sucrose | 65.8 | g | 32.9 | g |
| Glucose | 36 | g | 36 | g |

Autoclave

| | | |
|---|---|---|
| Acetosyringone (stock 20 ml/ml) | 1 | ml |

TABLE 6
10 X MSAA media

| | |
|---|---|
| 1 L | 200 ml |

| | | | | |
|---|---|---|---|---|
| CaCl₂•2H₂O | 4.4 | g | 0.88 | g |
| MgSO₄•7H₂O | 3.7 | g | 0.74 | g |
| KH₂PO₄ | 1.7 | g | 0.34 | g |
| L-Glutamine | 8.769 | g | 1.7538 | g |
| L-Aspartic acid | 2.662 | g | 0.5324 | g |
| L-Arginine | 1.762 | g | 0.3524 | g |
| Glycine | 0.075 | g | 0.0150 | g |

Autoclave

Media for 1^{st} Selection of Callus

2N6 media 1L—autoclave

Hygromycin (50 mg/ml) 200 ul (final concentration 150 mg/L)

Cephatoxin (250 mg/ml) 1 ml (final concentration 1650 mg/L)

Media for 2^{nd} Selection of Callus

2N6 media 1L—autoclave

Hygromycin (50 mg/ml) 600 ul

Cephatoxin (250 mg/ml) 1 ml

TABLE 7
Media for 1^{st} redifferentiation of callus

| | 1 L | | 250 ml | | |
|---|---|---|---|---|---|
| MS | 4.3 | g | 1.075 | g | Sigma M5524(10 L) |
| MS Vitamin | 1 | ml | 250 | ul | |
| Sucrose | 30 | g | 7.5 | g | |
| Sorbitol | 30 | g | 7.5 | g | |
| Casamino acid | 2 | g | 0.5 | g | |
| MES | 11 | g | 2.75 | g | Acros 172595000 |

pH 5.8

| | | | | |
|---|---|---|---|---|
| Gellan gum | 4 | g | 1 | g |

After autoclave

| | | | | | |
|---|---|---|---|---|---|
| NAA (2 mg/ml) | 20 | ul | 5 | ul | |
| kinetin (1 mg/ml) | 10 | ul | 2.5 | ul | Sigma |
| Cephatoxin | 1 | ml | 25. | ul | Bioworld |
| Hygromycin | 600 | ul | 150 | ul | |

TABLE 8
Media for 2^{nd} redifferentiation of callus

| | |
|---|---|
| 1 L | 500 ml |

| | | | | |
|---|---|---|---|---|
| MS | 4.3 | g | 2.15 | g |
| MS vitamin | 1 | ml | 5 | ul |
| Sucrose | 30 | g | 15 | g |

pH 5.8

| | | | | |
|---|---|---|---|---|
| Gellan gum | 4 | g | 2 | g |

After autoclave

TABLE 9
Final media (callus formation - ½ MS media (using a bottle))

| | 1 L | | 500 ml | | 250 ml | |
|---|---|---|---|---|---|---|
| MS | 2.15 | g | 1.07 | g | 0.54 | g |
| Sucrose | 15 | g | 7.5 | g | 3.75 | g |
| Gellan gum (phyta gel) | 4(3) | g | 2(1.5) | g | 1(0.75) | g |

FIG. 17 shows the results of the second selection using hygromycin (30 ug/ml) for the rice callus that has been transformed with vectors of pCamLA or pTflA, respectively. The culture plate on the right side indicates that the rice callus that has been transformed with pTflA vector of the present invention starts to die out.

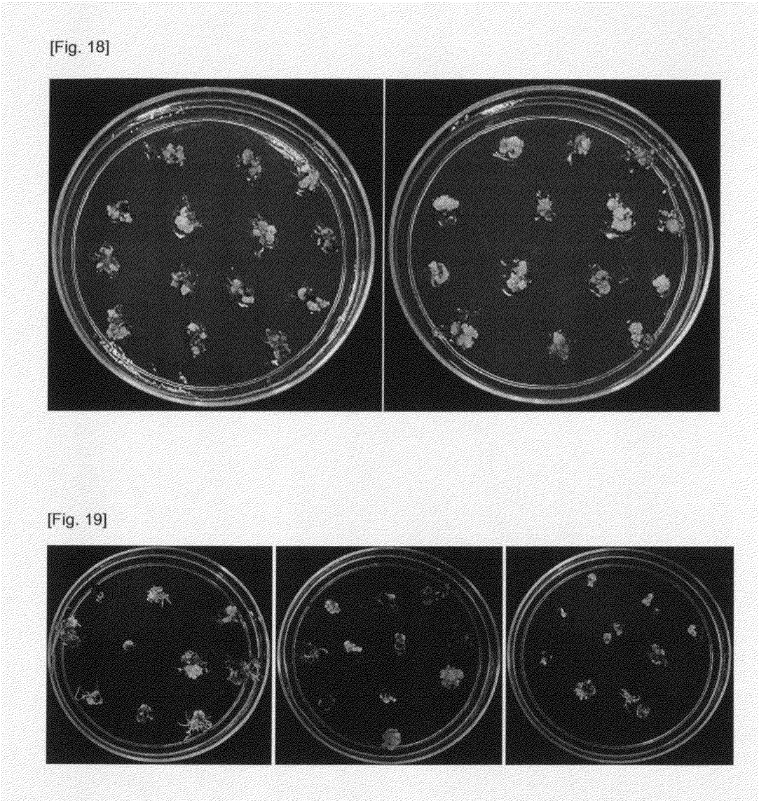

FIG. 18 shows the survival of callus in which selection with toxoflavin (5 ug/ml) was carried out before the redifferentiation of rice plants that have been transformed with either pCamLA or pTflA vector. Because the rice callus that has been transformed with pCamLA vector and placed in the media in the left side did not carry an enzyme which can degrade toxoflavin, most of the callus died out. However, for the rice callus transformed with pTflA vector and placed in the media in the right side, only a part of the callus died out.

FIG. 19 shows the survival of callus in which selection with toxoflavin (7.5 ug/ml) was carried out four weeks after the placement of the rice plants that, have been transformed with pCamLA vector on the medium for redifferentiation. As it is shown in the FIG. 19, because the rice callus transformed with pCamLA vector did not carry an enzyme which can degrade toxoflavin, most of the callus died out.

FIG. 20 shows the survival of callus in which selection with toxoflavin (7.5 ug/ml) was carried out four weeks after the placement of the rice plants that have been transformed with pTflA vector on the medium for redifferentiation. As it is shown in the FIG. 20, because the rice callus transformed with pTflA vector carried an enzyme which can degrade toxoflavin, the callus was redifferentiated normally.

Redifferentiation ratio in accordance with the selection with toxoflavin (7.5 ug/ml) for the rice callus, that had been each transformed with either pCamLA or pTflA vector, was determined four weeks after its placement on the redifferentiation media. Results are summarized in the following Table 10.

TABLE 10
Redifferentiation ratio

| | pCamLA(hpt) | pTflA |
|---|---|---|
| 2^{nd} transformation | 2/11(18.18%) | 28/84(33.3%) |
| 3^{rd} transformation | 11/109(10.09%) | 26/192(13.54%) |

In Table 10 above, redifferentiation ratio indicates the ratio of the number of redifferentiated plants compared to total number of callus. As it is clear from the result of Table 10, the redifferentiation ratio was higher in pTflA compared to pCamLA.

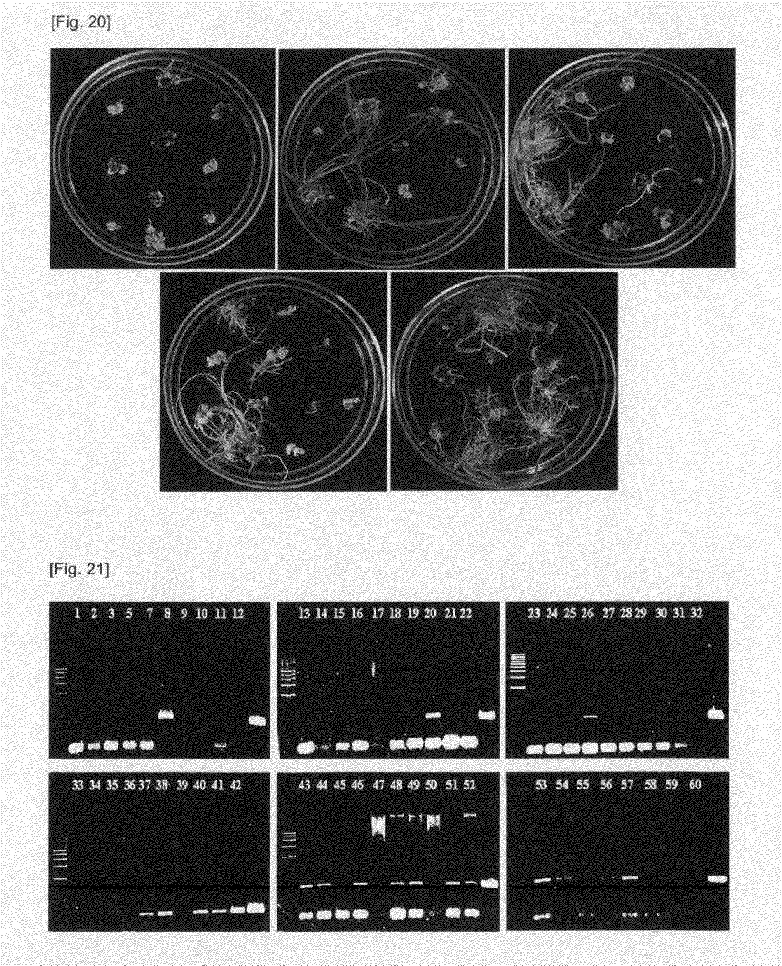

Selected plants were transplanted to a pot and their transformation with tflA gene was confirmed by PCR analysis of their tissue samples. PCR primer was designed based on the sequence of tflA gene. Annealing temperature was 55□. For PCR primer, both of tfla140-U (5′-TGCAGCTGCTGATGGAACAAA-3′; SEQ ID NO: 13) and TFLA370-L (5′-TTATCCAGTACAGGTGCAGCT-3′; SEQ ID NO: 14) were used. FIG. 21 shows the result of an agarose electrophoresis of PCR product obtained from PCR of the genomic DNA which has been isolated from the selected transgenic plants. In FIG. 21, lanes 12, 16, 17, 18, 21 and 36 indicate no production of PCR product. As it is indicated in FIG. 21, the transgenic plants produced the PCR products at desired position, thus supporting that toxoflavin-degrading gene of the present invention has been stably incorporated to the genome of the rice plant.

### Example 11

### Transformation of Arabidopsis thaliana

1) Comparative Analysis of the Existing Transformation Vector and a New Transformation Vector Based on tflA

Comparative analysis of the existing transformation vector and a new transformation vector based on tflA was carried out. Specifically, pMBP1:tflA in which genes resistant to toxoflavin and hygromycin are comprised in, pCamLA(Δhpt):tflA in which a gene resistant to hygromycin is deleted, and pMBP1:tflA in which tflA is inserted to a binary vector pMBP1 are compared with pMBP1 that has been commonly used as a vector for transformation.

2) Experimental Method

Arabidopsis Col-0 was transformed with Agrobacterium comprising each of the constructs. Detailed method for the transformation is described below.
1. Arabidopsis Col-0 was grown in a pot until blooming. When flower stems start to develop and flowers are blooming, the flower stems were cut off with scissors.
2. Agrobacterium comprising the gene to be transformed were cultured overnight in LB broth.
3. Cultured Agrobacterium was centrifuged and suspended in 5% sucrose till O.D.=0.8. 0.03% silwet was added to the suspension.
4. One week after cutting off the flower stems, a newly grown flower stem was immersed in said 5% sucrose suspension for 2 to 3 seconds.
5. The plants were wrapped with a plastic bag. Bags were removed two days later.

After harvesting all the seeds, the constructs carrying tflA resistant gene were spread on MS plate containing toxoflavin with concentration of 20 uM (3.84 μg/Ml). For pMB1 carrying kanamycin resistant gene seeds were spread on MS plate containing kanamycin with concentration of 50 μg/Ml. Ten days later, transgenic organisms were selected from the plates. Approximately one thousand seeds were spread on each plate, and on average about 10 transgenic organisms were obtained from each plate.

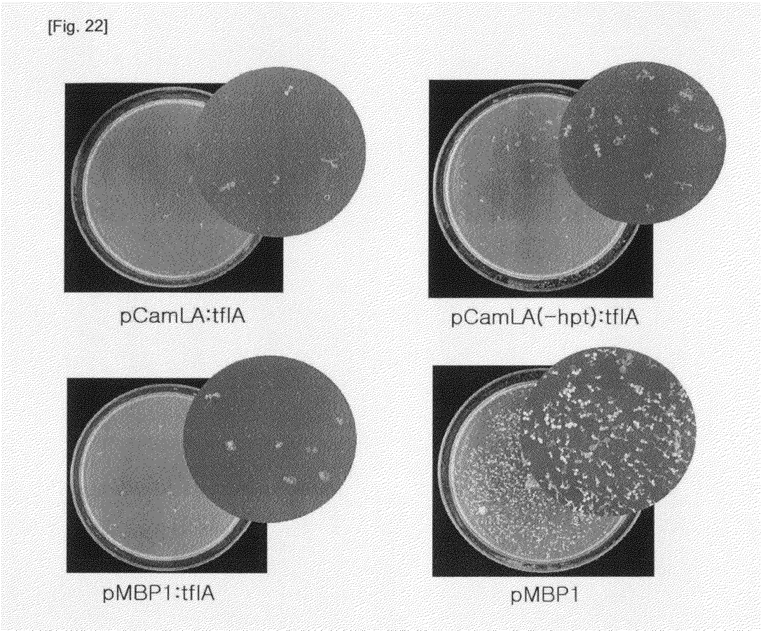

FIG. 22 includes the photo images taken for the transgenic plants to which vectors of pCamLA:tflA, pCamLA (Δhpt):tflA, pMBP1:tflA or pMBP1 have been transformed. The transgenic plants to which the toxoflavin-resistant gene has been incorporated show normal growth in the media comprising 20 uM toxoflavin. Roots were also growing well. However, germination did not occur for most of the non-transgenic organisms and even when the germination occurred, root growth was not normal. The level of the transformation with the vectors of the present invention appears to be similar to that of pMBP1 vector which has been widely used.

3) PCR and a Photo-Bleaching Test of Selected Transgenic Organisms

(1) PCR of Selected Transgenic Organisms

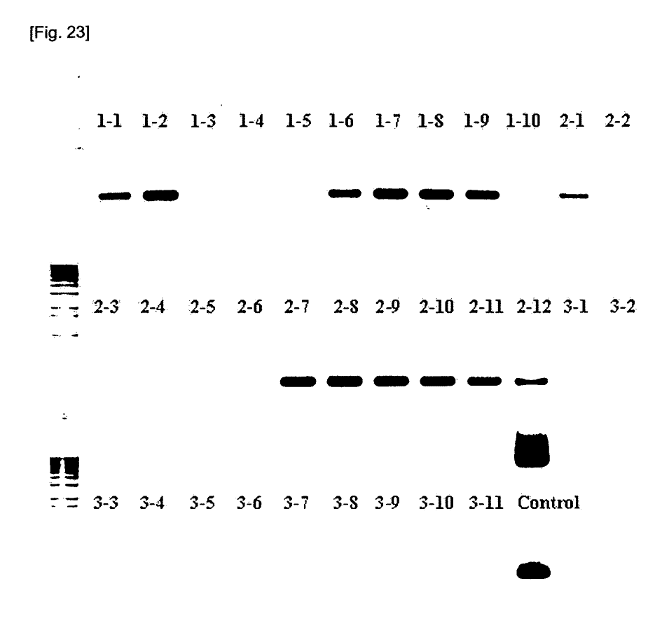

Some of the transgenic plants were selected and transferred to a pot. PCR was carried out to determine the transformation of the plants with tflA gene. PCR primer used for the reaction was designed based on the sequence of tflA gene. Annealing was carried out at the temperature of 55□. Two PCR primers used for the reaction are as follows: tfla140-U (5′-TGCAGCTGCTGATGGAACAAA-3′; SEQ ID NO: 13) and TFLA370-L (5′-TTATCCAGTACAGGTGCAGCT-3′; SEQ ID NO: 14). FIG. 23 shows the result of an agarose gel electrophoresis for the PCR product obtained from the PCR of the genomic DNA which has been isolated from the selected transgenic organisms. PCR analysis of the transgenic organisms which comprise the constructs of pCamLA:tflA (1-1˜1-10), pCamLA(□hpt):tflA (2-1˜2-12) or pMBP1:tflA (3-1˜3-11) shows that, the transgenic plants which germinated successfully in the media comprising toxoflavin and grew their roots well correspond to the same band as the control (i.e., tflA gene). On the other hand, for the plants of which root growth was not normal, no such band was observed. Therefore, it was confirmed that toxoflavin could be used as a selection marker for the transgenic plants.

(2) Identification of the Transgenic Organisms Based on a Photo-Bleaching Test

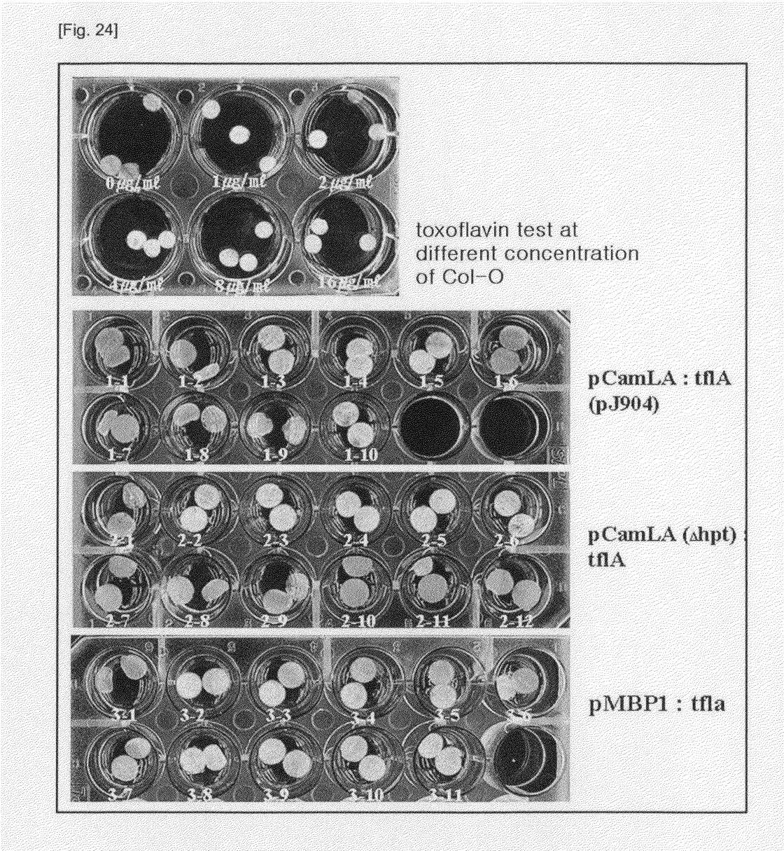

Toxoflavin, which is a component responsible for the pathogenic property of rice grain rot, results in a light-dependent bleaching when it is applied to plants, and such phenomenon occurs generally for all kinds of plants. In the present example, toxoflavin having various concentrations was tested against Arabidopsis Col-0. It was found that even at low concentration toxoflavin exerts a bleaching effect on the plant. On the basis of such bleaching effect by toxoflavin, the transgenic organisms that have been prepared according to the present invention were tested. FIG. 24 shows the results of the bleaching test for the transgenic organisms and the non-transgenic organisms. In the case of the transgenic organisms selected for the test, photo-bleaching was not observed. However, the bleaching occurred for the non-transgenic organisms. Such results correspond to the result of PCR described above. Therefore, compared to a previous marker selection system which is based on the use of antibiotics, the selection method of the present invention which utilizes toxoflavin as a selection marker is advantageous in that a system for preparing transgenic plants with a desired gene can be achieved without using any antibiotics.

### EFFECT OF THE INVENTION

According to the present invention, the transgenic plants which have been engineered to express tflA protein become to have resistance to toxoflavin. Especially for rice, such transgenic plants will have resistance to bacterial grain rot. Thus, increase in production amount and an improvement in quality of rice are expected. Additionally, selection of the transgenic rice plants can be carried out using toxoflavin, which is economically favorable compared to expensive antibiotics that have been used in prior art.

1.-21. (canceled)

22. An expression cassette of selection marker for plant transformation, comprising the following sequences that are operably linked in a 5′ to 3′ direction:
(i) a promoter sequence;
(ii) a coding sequence for an enzyme which degrades toxoflavin; and
(iii) a 3′-untranslated terminator sequence.

23. The expression cassette according to claim 22, further comprising an expression cassette for a target protein which comprises the following:
(i) a promoter sequence;
(ii) a coding sequence for the target protein; and
(iii) a 3′-untranslated terminator sequence.

24.-26. (canceled)

27. The expression cassette according to claim 22, wherein said coding sequence for the enzyme which degrades toxoflavin comprises the nucleotide sequence of SEQ ID NO: 1.

28. The expression cassette according to claim 22, wherein said coding sequence for the enzyme which degrades toxoflavin comprises a nucleotide sequence having sequence homology of at least 90% compared to the nucleotide sequence of SEQ ID NO: 1.

29. A recombinant vector comprising the expression cassette according to claim 22.

30. A host cell transformed with the recombinant vector of claim 29.

31. (canceled)

32. A plant transformed with the recombinant vector of claim 29.

33. The plant according to claim 32, wherein said plant is a rice plant or Arabidopsis thaliana.

34. Transgenic seeds of the plant according to claim 33.

35. A method of selecting a transgenic plant, comprising the steps of:
transforming a plant, a part of plant, or plant cells with the recombinant vector of claim 29 to produce a transgenic organism; and
proliferating the transgenic organism in a media comprising toxoflavin.

36. (canceled)

37. A method of producing a transgenic plant, comprising the steps of:
transforming plant cells with the recombinant vector of claim 29 to produce transgenic plant cells;
proliferating said transgenic plant cells in a media comprising toxoflavin to produce selected transgenic plant cells; and
growing a transgenic plant from said selected transgenic plant cells.
